(19) United States
(12) Patent Application Publication
(10) Pub. No.: US 2023/0142404 A1
(43) Pub. Date: May 11, 2023

(54) MEDICAL IMAGING APPARATUS, LEARNING MODEL GENERATION METHOD, AND LEARNING MODEL GENERATION PROGRAM

(71) Applicant: Sony Group Corporation, Tokyo (JP)

(72) Inventors: Yuka ARIKI, Tokyo (JP); Masaru USUI, Tokyo (JP); Daisuke NAGAO, Tokyo (JP); Kazuto YOKOYAMA, Tokyo (JP); Yohei KURODA, Tokyo (JP)

(73) Assignee: Sony Group Corporation, Tokyo (JP)

(21) Appl. No.: 17/916,045

(22) PCT Filed: Sep. 8, 2021
(86) PCT No.: PCT/JP2021/033054

§ 371 Date: Sep. 30, 2022

(30) Foreign Application Priority Data
Sep. 8, 2020 (JP) 2020-150815

(51) Int. Cl. A61B 1/00; G06N 20/00

(52) U.S. Cl. CPC A61B 1/00006; A61B 1/00149; G06N 20/00

(57) ABSTRACT

Systems and methods can comprise or involve predicting future movement information for a medical articulating arm using a learned model generated based on learned previous movement information from a prior non-autonomous trajectory of the medical articulating arm performed in response to operator input and using current movement information for the medical articulating arm, generating control signaling to autonomously control movement of the medical articulating arm in accordance with the predicted future movement information for the medical articulating arm, and autonomously controlling the movement of the medical articulating arm in accordance with the predicted future movement information for the medical articulating arm based on the generated control signaling.

### TECHNICAL FIELD

The present disclosure relates to a medical imaging apparatus, a learning model generation method, and a learning model generation program.

### BACKGROUND

In recent years, in endoscopic surgery, surgery has been performed while imaging in the abdominal cavity of a patient using an endoscope and displaying an image captured by the endoscope on a display. In such a case, it has been common for the endoscope to be operated by, for example, a surgeon or an assistant in accordance with the surgeon's instructions, to adjust the imaging range with the captured image so that a surgical site is properly displayed on the display. In such an endoscopic surgery, the burden on the surgeon can be reduced by enabling the autonomous operation of an endoscope. Patent Literatures 1 and 2 describe techniques applicable to the autonomous operation of an endoscope.

### CITATION LIST

### Patent Literature

PTL 1: JP 2017-177297 A

PTL 2: JP 6334714 B2

### SUMMARY

### Technical Problem

With regard to autonomous operation of an endoscope, for example, a method of measuring only an endoscope operation in response to a surgeon or an instruction of the surgeon and reproducing the measured endoscope operation can be considered. However, the method may cause a deviation between an image captured by the re-produced endoscope operation and an imaging range required for an actual surgery. Although a heuristic method of moving the endoscope to the center point of the tool position used by the surgeon has been also considered, the heuristic method has been often evaluated as unnatural by the surgeon.

The present disclosure aims to provide a medical imaging apparatus, a learning model generation method, and a learning model generation program that enable autonomous operation of an endoscope to be performed more appropriately.

### Solution to Problem

For solving the problem described above, a medical imaging apparatus according to one aspect of the present disclosure has an arm unit in which a plurality of links is connected by a joint unit and that supports an imaging unit that images a surgical field image; and a control unit that drives the joint unit of the arm unit based on the surgical field image to control a position and/or posture of the imaging unit, wherein the control unit has a learning unit that generates a learned model in which a trajectory of the position and/or posture is learned based on operations to the position and/or posture of the imaging unit, and that predicts the position and/or posture of the imaging unit using the learned model; and a correction unit that learns the trajectory based on a result of evaluation by a surgeon for the position and/or posture of the imaging unit driven based on the prediction.

### BRIEF DESCRIPTION OF DRAWINGS

FIG. 1 is a diagram schematically illustrating an example of a configuration of an endoscopic surgery system applicable to an embodiment of the present disclosure.

FIG. 2 is a block diagram illustrating an example of a functional configuration of a camera head and a CCU applicable to the embodiment.

FIG. 3 is a schematic view illustrating an external appearance of an example of a support arm apparatus applicable to the embodiment.

FIG. 4 is a schematic diagram illustrating a configuration of a forward-oblique viewing endoscope applicable to the embodiment.

FIG. 5 is a schematic diagram illustrating the forward-oblique viewing endoscope and a forward-viewing endoscope in contrast.

FIG. 6 is a diagram illustrating a configuration of an example of a robot arm apparatus applicable to the embodiment.

FIG. 7 is a functional block diagram of an example for explaining a function of a medical imaging system according to the embodiment.

FIG. 8 is a block diagram illustrating a configuration of an example of a computer capable of implementing a control unit according to the embodiment.

FIG. 9 is a functional block diagram of an example for explaining a function of a learning/correction unit according to the embodiment.

FIG. 10A is a diagram illustrating an example of a captured image captured by an endoscope device.

FIG. 10B is a diagram illustrating an example of a captured image captured by the endoscope device.

FIG. 11 is a schematic diagram for explaining the control of an arm unit according to the embodiment.

FIG. 12A is a schematic diagram for schematically explaining processing by a learning unit according to the embodiment.

FIG. 12B is a schematic diagram for schematically explaining processing by the learning unit according to the embodiment.

FIG. 13A is a schematic diagram for schematically explaining processing by a correction unit according to the embodiment.

FIG. 13B is a schematic diagram for schematically explaining processing by the correction unit according to the embodiment.

FIG. 14 is a schematic diagram for explaining the learning processing in the learning unit according to the embodiment.

FIG. 15 is a schematic diagram for explaining an example of a learning model according to the embodiment.

FIG. 16 is a flowchart illustrating an example of processing by the learning/correction unit according to the embodiment.

FIG. 17A is a diagram schematically illustrating a surgery using an endoscope system according to an existing technique.

FIG. 17B is a diagram schematically illustrating a surgery performed using a medical imaging system according to the embodiment is applied.

FIG. 18 is a flowchart illustrating an example of operations associated with the surgery performed using the medical imaging system according to the embodiment.

FIG. 19 is a functional block diagram illustrating an example of a functional configuration of a medical imaging system corresponding to a trigger signal outputted by voice applicable to the embodiment.

### DESCRIPTION OF EMBODIMENTS

Embodiments of the present disclosure will be described below in detail based on the drawings. In the following embodiments, the same reference numerals are assigned to the same portions, and the description thereof is omitted.

The embodiments of the present disclosure will be described below in the following order.

1. Techniques Applicable to Embodiment of Present Disclosure

1-1. Configuration Example of Endoscopic Surgery System Applicable to Embodiment

1-2. Specific Configuration Example of Support Arm Apparatus

1-3. Basic Configuration of Forward-Oblique Viewing Endoscope

1-4. Configuration Example of Robot Arm Apparatus Applicable to Embodiment

2. Embodiment of Present Disclosure

2-1. Overview of Embodiment

2-2. Configuration Example of Medical Imaging System according to Embodiment

2-3. Overview of Processing by Medical Imaging System according to Embodiment

2-4. Details of Processing by Medical Imaging System according to Embodiment

2-4-1. Processing of Learning Unit according to Embodiment

2-4-2. Processing of Correction Unit according to Embodiment

2-4-3. Overview of Surgery when Medical Imaging System according to Embodiment is Applied

2-5. Variation of Embodiment

2-6. Effect of Embodiment

2-7. Application Example of Techniques of Present Disclosure

### 1. Techniques Applicable to Embodiment of Present Disclosure

Prior to the description of embodiments of the present disclosure, techniques applicable to the embodiments of the present disclosure will be first described for ease of understanding.

### 1-1. Configuration Example of Endoscopic Surgery System Applicable to Embodiment

### Overview of Endoscopic Surgery System

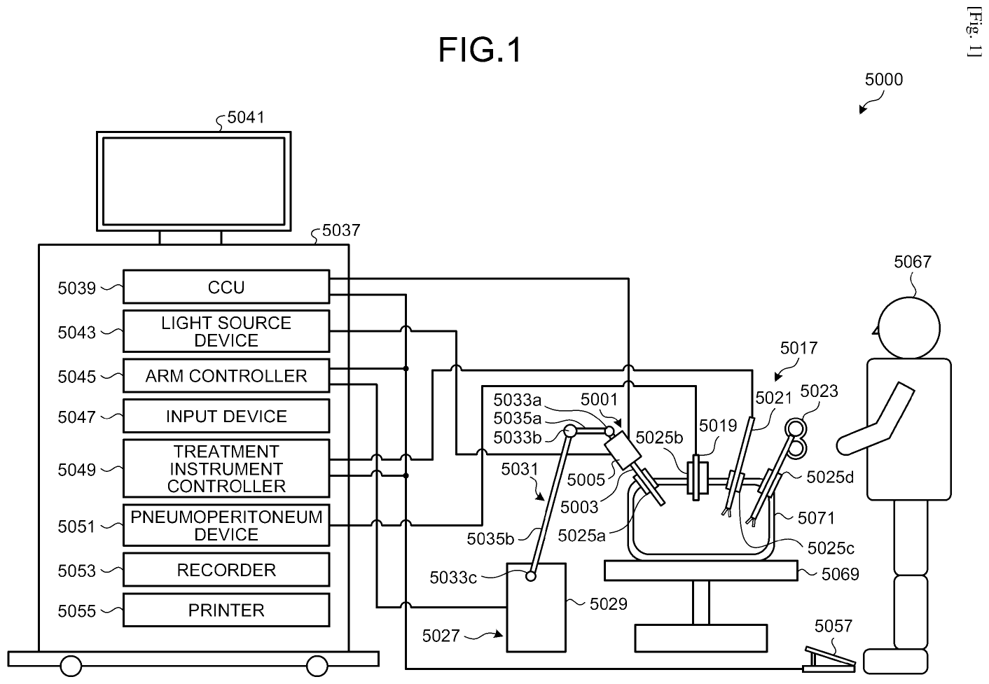

FIG. 1 is a diagram schematically illustrating an example of a configuration of an endoscopic surgery system 5000 applicable to an embodiment of the present disclosure. FIG. 1 illustrates a surgeon (physician) 5067 using the endoscopic surgery system 5000 to perform surgery on a patient 5071 on a patient bed 5069. In the example of FIG. 1, the endoscopic surgery system 5000 includes an endoscope 5001, other surgical instruments 5017, a support arm apparatus 5027 for supporting the endoscope 5001, and a cart 5037 on which various devices for endoscopic surgery are mounted.

In endoscopic surgery, instead of cutting through and opening the abdominal wall, the abdominal wall is punctured with multiple cylindrical tools called trocars 5025a to 5025d. From the trocars 5025a to 5025d, a lens barrel 5003 of the endoscope 5001 and the other surgical instruments 5017 are inserted into the body cavity of the patient 5071.

In the example of FIG. 1, a pneumoperitoneum tube 5019, an energy treatment instrument 5021, and forceps 5023 are inserted into the body cavity of the patient 5071 as the other surgical instruments 5017. The energy treatment instrument 5021 is a treatment instrument for, for example, cutting and peeling a tissue or sealing a blood vessel by a high-frequency current or ultrasonic vibration. However, the surgical instrument 5017 illustrated in FIG. 1 is merely an example, and as the surgical instrument 5017, various surgical instruments generally used in endoscopic surgery, such as tweezers and a retractor, may be used.

An image of the surgical site in the body cavity of the patient 5071 captured by the endoscope 5001 is displayed on a display device 5041. The surgeon 5067 performs a treatment such as cutting the affected part by using the energy treatment instrument 5021 or forceps 5023 while viewing an image of the surgical site displayed on the display device 5041 in real time. Although not illustrated, the pneumoperitoneum tube 5019, the energy treatment instrument 5021, and the forceps 5023 are supported by, for example, the surgeon 5067 or an assistant during surgery.

### Support Arm Apparatus

The support arm apparatus 5027 includes an arm unit 5031 extending from a base unit 5029. In the example of FIG. 1, the arm unit 5031 is constituted of joint units 5033a, 5033b, and 5033c, and links 5035a and 5035b, and is driven by control from an arm controller 5045. The arm unit 5031 supports the endoscope 5001 and controls its position and/or posture. Thus, the endoscope 5001 can be fixed at a stable position.

The position of the endoscope indicates the position of the endoscope in space, and can be expressed as a three-dimensional coordinate such as a coordinate (x, y, z). Further, the posture of the endoscope indicates the direction in which the endoscope faces, and can be expressed as a three-dimensional vector, for example.

### Endoscope

The endoscope 5001 will be described schematically. The endoscope 5001 is constituted of the lens barrel 5003 in which a region of a predetermined length from its tip is inserted into the body cavity of the patient 5071, and a camera head 5005 connected to the base end of the lens barrel 5003. In the illustrated example, although the endoscope 5001 configured as a so-called rigid endoscope having a rigid lens barrel 5003 is illustrated, the endoscope 5001 may be configured as a so-called flexible endoscope having a flexible lens barrel 5003.

An opening into which an objective lens is fitted is provided at the tip of the lens barrel 5003. The endoscope 5001 is connected to a light source device 5043 mounted on the cart 5037, and the light generated by the light source device 5043 is guided to the tip of the lens barrel 5003 by a light guide extended inside the lens barrel, and is emitted toward an observation target in the body cavity of the patient 5071 through an objective lens. Note that the endoscope 5001 may be a forward-viewing endoscope, a forward-oblique viewing endoscope, or a side-viewing endoscope.

An optical system and an imaging element are provided inside the camera head 5005, and reflected light (observation light) from an observation target is condensed on the imaging element by the optical system. The observation light is photoelectrically converted by the imaging element, and an electric signal corresponding to the observation light, that is, an image signal corresponding to the observation image is generated. The image signal is transmitted as RAW data to a camera control unit (CCU) 5039. The camera head 5005 has a function of adjusting the magnification and the focal length by appropriately driving the optical system.

In order to support stereoscopic viewing (3D display), for example, the camera head 5005 may be provided with a plurality of imaging elements. In this case, a plurality of relay optical systems is provided inside the lens barrel 5003 in order to guide observation light to each of the plurality of imaging elements.

### Various Devices Mounted on Cart

In the example of FIG. 1, the cart 5037 is mounted with the CCU 5039, the light source device 5043, the arm controller 5045, an input device 5047, a treatment instrument controller 5049, a pneumoperitoneum device 5051, a recorder 5053, and a printer 5055.

The CCU 5039 is constituted of, for example, a central processing unit (CPU) and a graphics processing unit (GPU), and integrally controls operations of the endoscope 5001 and the display device 5041. Specifically, the CCU 5039 performs various image processing on the image signal received from the camera head 5005, such as development processing (demosaic processing), for displaying an image based on the image signal. The CCU 5039 provides the image signal subjected to the image processing to the display device 5041. The CCU 5039 also transmits control signals to the camera head 5005 to control its drive. The control signal may include information about imaging conditions such as magnification and focal length.

The display device 5041 displays an image based on the image signal subjected to image processing by the CCU 5039 under the control of the CCU 5039. When the endoscope 5001 is compatible with high-resolution imaging, such as 4K (3840 horizontal pixels×2160 vertical pixels) or 8K (7680 horizontal pixels×4320 vertical pixels), and/or 3D display, the display device 5041 may be one capable of high-resolution display and/or one capable of 3D display, respectively. In the case of a display device corresponding to high-resolution imaging such as 4K or 8K, a display device 5041 having a size of 55 inches or larger can provide a more immersive feeling. Further, a plurality of display devices 5041 different in resolution and size may be provided depending on the application.

The light source device 5043 includes a light emitting element such as a light emitting diode (LED) and a drive circuit for driving the light emitting element, and supplies irradiation light for imaging the surgical site to the endoscope 5001.

The arm controller 5045 includes, for example, a processor such as a CPU, and operates according to a predetermined program to control the drive of the arm unit 5031 of the support arm apparatus 5027 according to a predetermined control method.

The input device 5047 is an input interface to the endoscopic surgery system 5000. The user can input various types of information and instructions to the endoscopic surgery system 5000 through the input device 5047. For example, the user inputs various types of information related to the surgery, such as the physical information of the patient and the surgical procedure, through the input device 5047. Further, for example, through the input device 5047, the user inputs an instruction to drive the arm unit 5031, an instruction to change the imaging conditions (for example, type, magnification, and focal length of irradiation light) by the endoscope 5001, and an instruction to drive the energy treatment instrument 5021, for example.

The type of the input device 5047 is not limited, and the input device 5047 may be any of various known input devices. As the input device 5047, an input device such as a mouse, a keyboard, a touch panel, a switch, a lever, or a joystick can be applied. As the input device 5047, a plurality of types of input devices can be mixedly applied. A foot switch 5057 operated by the foot of the operator (for example, a surgeon) can also be applied as the input device 5047. When a touch panel is used as the input device 5047, the touch panel may be provided on the display surface of the display device 5041.

The input device 5047 is not limited to the above example. For example, the input device 5047 can be applied to a device worn by a user, such as a wearable device of a glasses-type or a head mounted display (HMD). In this case, the input device 5047 can perform various inputs according to the gestures and sight lines of the user detected by devices worn by the users.

The input device 5047 may also include a camera capable of detecting user movement. In this case, the input device 5047 can perform various inputs according to the gestures and sight lines of the user detected from the video captured by the camera. Further, the input device 5047 can include a microphone capable of picking up the voice of the user. In this case, various inputs can be performed by the voice picked up by the microphone.

Since the input device 5047 is configured to be able to input various types of information in a non-contact manner as described above, a user (for example, the surgeon 5067) belonging to a clean area in particular can operate a device belonging to a dirty area in a non-contact manner. Further, since the user can operate a device without releasing his/her hand from a surgical instrument, the convenience of the user is improved.

The treatment instrument controller 5049 controls the drive of the energy treatment instrument 5021 for tissue cauterization, incision, or blood vessel sealing, for example. The pneumoperitoneum device 5051 feeds gas into the body cavity of the patient 5071 through the pneumoperitoneum tube 5019 in order to inflate the body cavity of the patient 5071 for the purpose of securing the visual field by the endoscope 5001 and securing the working space of the surgeon. The recorder 5053 is a device that can record various types of information about surgery. The printer 5055 is a device that can print various types of information about surgery in various formats, such as text, images, or graphs.

A particularly characteristic configuration of the endoscopic surgery system 5000 will be described below in more detail.

### Support Arm Apparatus

The support arm apparatus 5027 includes the base unit 5029 being a base, and the arm unit 5031 extending from the base unit 5029. In the example of FIG. 1, the arm unit 5031 includes a plurality of joint units 5033a, 5033b, and 5033c, and a plurality of links 5035a and 5035b connected by the joint unit 5033b. In FIG. 1, the configuration of the arm unit 5031 is simplified for simplicity.

In practice, the shape, number, and arrangement of the joint units 5033a to 5033c and the links 5035a and 5035b, as well as the orientation of the axis of rotation of the joint units 5033a to 5033c may be appropriately set such that the arm unit 5031 has the desired degree of freedom. For example, the arm unit 5031 may be suitably configured to have six or more degrees of freedom. Thus, the endoscope 5001 can be freely moved within the movable range of the arm unit 5031, so that the lens barrel 5003 of the endoscope 5001 can be inserted into the body cavity of the patient 5071 from a desired direction.

The joint units 5033a to 5033c are provided with actuators, and the joint units 5033a to 5033c are configured to be rotatable about a predetermined rotation axis by driving the actuators. Controlling the drive of the actuators by the arm controller 5045 allows the rotation angle of each of the joint units 5033a to 5033c to be controlled and the drive of the arm unit 5031 to be controlled. Thus, the position and/or posture of the endoscope 5001 can be controlled. In this regard, the arm controller 5045 can control the drive of the arm unit 5031 by various known control methods such as force control or position control.

For example, the surgeon 5067 may appropriately input an operation via the input device 5047 (including the foot switch 5057), and the arm controller 5045 may appropriately control the drive of the arm unit 5031 according to the operation input, thereby controlling the position and/or the posture of the endoscope 5001. The control allows the endoscope 5001 at the tip of the arm unit 5031 to be moved from an arbitrary position to an arbitrary position and then to be fixedly supported at the position after the movement. The arm unit 5031 may be operated by a so-called master/slave mode. In this case, the arm unit 5031 (slave) may be remotely controlled by the user via the input device 5047 (master console) located remote from or within a surgical room.

Further, when force control is applied, the arm controller 5045 may perform so-called power assist control for driving the actuators of the joint units 5033a to 5033c so that the arm unit 5031 smoothly move according to external force applied from the user. Thus, when the user moves the arm unit 5031 while directly touching the arm unit 5031, the arm unit 5031 can be moved with a relatively light force. Therefore, enabling the endoscope 5001 to move more intuitively and with a simpler operation allows the convenience of the user to be improved.

In endoscopic surgery, the endoscope 5001 has been generally supported by a surgeon called a scopist. On the other hand, using the support arm apparatus 5027 allows the position of the endoscope 5001 to be fixed more reliably without manual operation, so that an image of the surgical site can be obtained stably and the surgery can be performed smoothly.

Note that the arm controller 5045 may not necessarily be provided on the cart 5037. Further, the arm controller 5045 may not necessarily be a single device. For example, the arm controller 5045 may be provided in each of the joint units 5033a to 5033c of the arm unit 5031 of the support arm apparatus 5027, and the plurality of arm controllers 5045 may cooperate with each other to realize the drive control of the arm unit 5031.

### Light Source Device

The light source device 5043 supplies the endoscope 5001 with irradiation light for imaging a surgical site. The light source device 5043 is constituted of a white light source constituted of, for example, an LED, a laser light source, or a combination thereof. In a case where the white light source is constituted by the combination of the RGB laser light sources, the output intensity and output timing of each color (each wavelength) can be controlled with high accuracy, so that the white balance of the captured image can be adjusted in the light source device 5043. In this case, the observation target is irradiated with laser light from each of the RGB laser light sources in time division, and the drive of the imaging element of the camera head 5005 is controlled in synchronization with the irradiation timing, so that images corresponding to each of the RGB can be imaged in time division. According to the method, a color image can be obtained without providing a color filter to the imaging element.

The drive of the light source device 5043 may also be controlled so as to change the intensity of the output light at predetermined intervals. Controlling the drive of the imaging element of the camera head 5005 in synchronization with the timing of the change of the intensity of the light to acquire images in time division, and synthesizing the images allows an image of a high dynamic range without so-called black collapse and white skipping to be generated.

The light source device 5043 may be configured to supply light of a predetermined wavelength band corresponding to special light observation. In the special light observation, for example, so-called narrow-band light observation (Narrow Band Imaging) is carried out, in which a predetermined tissue such as a blood vessel on the mucosal surface layer is imaged with high contrast by irradiating the tissue with light of a narrow-band compared to the irradiation light (i.e., white light) at the time of normal observation by utilizing the wavelength dependence of light absorption in the body tissue.

Alternatively, in the special light observation, fluorescence observation for obtaining an image by fluorescence generated by applying excitation light may be performed. In the fluorescence observation, for example, irradiating a body tissue with excitation light to observe the fluorescence from the body tissue (auto-fluorescence observation), or by locally injecting a reagent such as indocyanine green (ICG) into a body tissue and irradiating the body tissue with excitation light corresponding to a fluorescence wavelength of the reagent to obtain a fluorescent image can be performed.

The light source device 5043 can be configured to supply narrow band light and/or excitation light corresponding to such special light observation.

### Camera Head and CCU

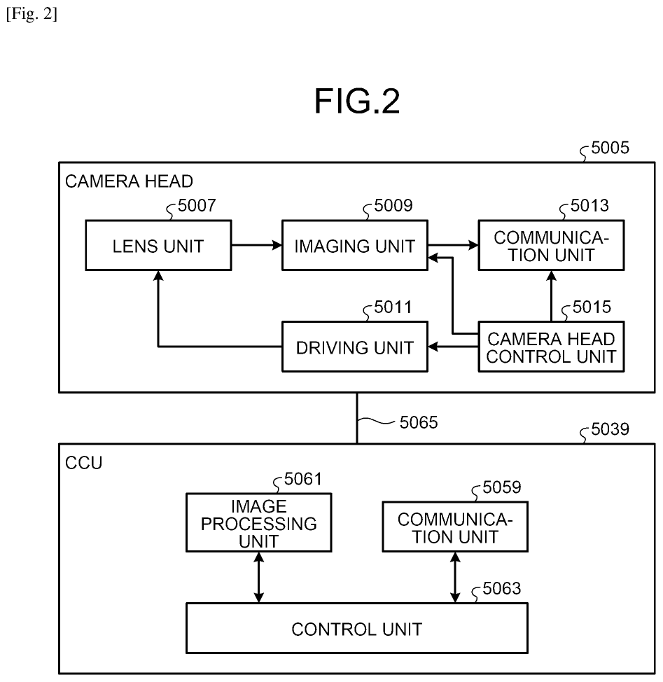

The functions of the camera head 5005 of the endoscope 5001 and the CCU 5039 will be described in more detail with reference to FIG. 2. FIG. 2 is a block diagram illustrating an example of a functional configuration of the camera head 5005 and the CCU 5039 illustrated in FIG. 1.

Referring to FIG. 2, the camera head 5005 includes, as its functions, a lens unit 5007, an imaging unit 5009, a driving unit 5011, a communication unit 5013, and a camera head control unit 5015. The CCU 5039 includes, as its functions, a communication unit 5059, an image processing unit 5061, and a control unit 5063. The camera head 5005 and the CCU 5039 are connected by a transmission cable 5065 so as to be communicable in both directions.

The functional configuration of the camera head 5005 will be first described. The lens unit 5007 is an optical system provided at a connection portion with the lens barrel 5003. The observation light taken in from the tip of the lens barrel 5003 is guided to the camera head 5005 and made incident on the lens unit 5007. The lens unit 5007 is constituted by combining a plurality of lenses including a zoom lens and a focus lens. The optical characteristics of the lens unit 5007 are adjusted so as to converge the observation light on the light receiving surface of the imaging element of the imaging unit 5009. Further, the zoom lens and the focus lens are configured so that the lenses positions on the optical axis can be moved for adjusting the magnification and the focus of the captured image.

The imaging unit 5009 is constituted of an imaging element, and is arranged at the rear stage of the lens unit 5007. The observation light passing through the lens unit 5007 is converged on the light receiving surface of the imaging element, and an image signal corresponding to the observation image is generated by photoelectric conversion. The image signal generated by the imaging unit 5009 is provided to the communication unit 5013.

The imaging element constituting the imaging unit 5009 is, for example, a complementary metal oxide semiconductor (CMOS) type image sensor in which color filters of R (red), G (green), and B (blue) colors are arranged in a Bayer array and which is capable of color imaging. The imaging element may be, for example, a device capable of taking an image of 4K or higher resolution. Obtaining the image of the surgical site at high resolution allows the surgeon 5067 to grasp the state of the surgical site in more detail, and the surgery to proceed more smoothly.

The imaging element constituting the imaging unit 5009 is configured to have a pair of imaging elements for acquiring image signals for the right eye and image signals for the left eye, respectively, corresponding to 3D display. Performing the 3D display allows the surgeon 5067 to more accurately grasp the depth of the biological tissue in the surgical site. In the case where the imaging unit 5009 is formed of a multi-plate type, a plurality of lens units 5007 is provided corresponding to the respective imaging elements.

Further, the imaging unit 5009 may not necessarily be provided in the camera head 5005. For example, the imaging unit 5009 may be provided inside the lens barrel 5003 immediately behind the objective lens.

The driving unit 5011 is constituted of an actuator and moves the zoom lens and the focus lens of the lens unit 5007 by a predetermined distance along the optical axis under the control of the camera head control unit 5015. Thus, the magnification and focus of the captured image by the imaging unit 5009 can be appropriately adjusted.

The communication unit 5013 is constituted of a communication device for transmitting and receiving various types of information to and from the CCU 5039. The communication unit 5013 transmits the image signal obtained from the imaging unit 5009 as RAW data via the transmission cable 5065 to the CCU 5039. In this regard, the image signal is preferably transmitted by optical communication in order to display the captured image of the surgical site with low latency. The optical communication transmission is because the surgeon 5067 performs surgery while observing the condition of the affected part by the captured image during surgery, so that the moving image of the surgical site is required to be displayed in real time as much as possible for safer and more reliable surgery. When optical communication is performed, the communication unit 5013 is provided with a photoelectric conversion module for converting an electric signal into an optical signal. The image signal is converted into an optical signal by the photoelectric conversion module and then transmitted through the transmission cable 5065 to the CCU 5039.

Further, the communication unit 5013 receives, from the CCU 5039, a control signal for controlling the drive of the camera head 5005. The control signal includes information relating to imaging conditions such as information for specifying a frame rate of a captured image, information for specifying an exposure value at the time of imaging, and/or information for specifying a magnification and a focus of the captured image. The communication unit 5013 provides the received control signal to the camera head control unit 5015. The control signal from the CCU 5039 may also be transmitted by optical communication. In this case, the communication unit 5013 is provided with a photoelectric conversion module for converting an optical signal into an electric signal, and the control signal is converted into an electric signal by the photoelectric conversion module and then provided to the camera head control unit 5015.

The imaging conditions such as the frame rate, the exposure value, the magnification and the focus are automatically set by the control unit 5063 of the CCU 5039 based on the acquired image signal. In other words, so-called auto exposure (AE) function, auto focus (AF) function, and auto white balance (AWB) function are mounted on the endoscope 5001.

The camera head control unit 5015 controls the drive of the camera head 5005 based on the control signal from the CCU 5039 received through the communication unit 5013. For example, the camera head control unit 5015 controls the drive of the imaging element of the imaging unit 5009 based on the information for specifying the frame rate of the captured image and/or the information for specifying the exposure at the time of imaging. Further, for example, the camera head control unit 5015 appropriately moves the zoom lens and the focus lens of the lens unit 5007 through the driving unit 5011 based on the information for specifying the magnification and the focus of the captured image. The camera head control unit 5015 may further include a function for storing information for identifying the lens barrel 5003 and the camera head 5005.

Arranging, for example, the lens unit 5007 and the imaging unit 5009 in a sealed structure having high airtightness and waterproofness allows the camera head 5005 to be made resistant to autoclave sterilization.

The functional configuration of the CCU 5039 will be then described. The communication unit 5059 is constituted of a communication device for transmitting and receiving various types of information to and from the camera head 5005. The communication unit 5059 receives an image signal transmitted from the camera head 5005 via the transmission cable 5065. In this regard, as described above, the image signal can be suitably transmitted by optical communication. In this case, the communication unit 5059 is provided with a photoelectric conversion module for converting an optical signal into an electric signal in correspondence with optical communication. The communication unit 5059 provides an image signal converted into an electric signal to the image processing unit 5061.

The communication unit 5059 transmits a control signal for controlling the drive of the camera head 5005 to the camera head 5005. The control signal may also be transmitted by optical communication.

The image processing unit 5061 applies various image processing to an image signal being RAW data transmitted from the camera head 5005. The image processing includes, for example, development processing and high-quality image processing. The high-quality image processing may include, for example, one or more of the processes such as band enhancement processing, super-resolution processing, noise reduction (NR) processing, and camera shake correction processing. The image processing may also include various known signal processing such as enlargement processing (electronic zoom processing). Further, the image processing unit 5061 performs detection processing on the image signal for performing AE, AF and AWB.

The image processing unit 5061 is constituted by a processor such as a CPU or a GPU, and the above-described image processing and detection processing can be performed by operating the processor according to a predetermined program. In the case where the image processing unit 5061 is constituted by a plurality of GPUs, the image processing unit 5061 appropriately divides information relating to image signals, and these GPUs perform image processing in parallel.

The control unit 5063 performs various controls related to the imaging of the surgical site by the endoscope 5001 and the display of the captured image. For example, the control unit 5063 generates a control signal for controlling the drive of the camera head 5005. In this regard, when the imaging condition is inputted by the user, the control unit 5063 generates a control signal based on the input by the user. Alternatively, when the endoscope 5001 is equipped with an AE function, an AF function, and an AWB function, the control unit 5063 appropriately calculates an optimum exposure value, a focal length, and a white balance in accordance with the result of detection processing by the image processing unit 5061, and generates a control signal.

Further, the control unit 5063 causes the display device 5041 to display an image of the surgical site based on the image signal subjected to the image processing by the image processing unit 5061. In this regard, the control unit 5063 uses various image recognition techniques to recognize various objects in the image of the surgical site. For example, the control unit 5063 can recognize a surgical instrument such as a forceps, a specific living body part, bleeding, or mist when using the energy treatment instrument 5021, for example, by detecting the shape and color of the edge of the object included in the image of the surgical site. The control unit 5063 superimposes and displays various types of surgery support information on the image of the surgical site by using the recognition result when displaying the image of the surgical site on the display device 5041. The surgery support information is superimposed and displayed and presented to the surgeon 5067, so that the surgery can be performed more safely and reliably.

The transmission cable 5065 connecting the camera head 5005 and the CCU 5039 is an electric signal cable corresponding to the communication of an electric signal, an optical fiber corresponding to the optical communication, or a composite cable thereof.

In the illustrated example, although communication is performed by wire using the transmission cable 5065, communication between the camera head 5005 and the CCU 5039 may be performed by wireless. In the case where the communication between the camera head 5005 and the CCU 5039 is performed by wireless, installing the transmission cable 5065 in the surgical room is not required, and thus the situation that the movement of the medical staff in the surgical room is prevented by the transmission cable 5065 can be eliminated.

An example of the endoscopic surgery system 5000 to which the technique of the present disclosure may be applied has been described above. Although the endoscopic surgery system 5000 has been described herein as an example, the system to which the technique of the present disclosure may be applied is not limited to such an example. For example, the techniques of the present disclosure may be applied to flexible endoscopic systems for testing and microsurgical systems.

### 1-2. Specific Configuration Example of Support Arm Apparatus

An example of a more specific configuration of the support arm apparatus applicable to the embodiment will be then described. Although the support arm apparatus described below is an example configured as a support arm apparatus for supporting the endoscope at the tip of the arm unit, the embodiment is not limited to the example. Further, when the support arm apparatus according to the embodiment of the present disclosure is applied to the medical field, the support arm apparatus according to the embodiment of the present disclosure can function as a medical support arm apparatus.

### External Appearance of Support Arm Apparatus

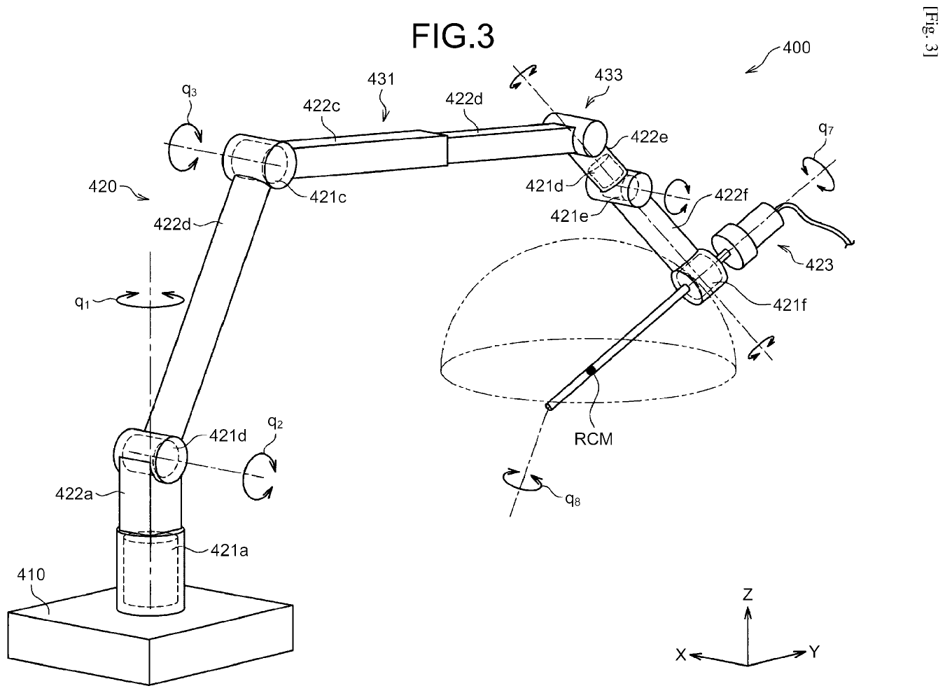

A schematic configuration of a support arm apparatus 400 applicable to the embodiment of the present disclosure will be first described with reference to FIG. 3. FIG. 3 is a schematic view illustrating an external appearance of an example of the support arm apparatus 400 applicable to the embodiment. The support arm apparatus 400 illustrated in FIG. 3 can be applied to the support arm apparatus 5027 described with reference to FIG. 1.

The support arm apparatus 400 illustrated in FIG. 3 includes a base unit 410 and an arm unit 420. The base unit 410 is a base of the support arm apparatus 400, and the arm unit 420 is extended from the base unit 410. Although not illustrated in FIG. 3, a control unit that integrally controls the support arm apparatus 400 may be provided in the base unit 410, and the drive of the arm unit 420 may be controlled by the control unit. The control unit is constituted by various signal processing circuits such as a CPU and a digital signal processor (DSP).

The arm unit 420 has a plurality of active joint units 421a to 421f, a plurality of links 422a to 422f, and an endoscope device 423 as a leading end unit provided at the tip of the arm unit 420.

The links 422a to 422f are substantially rod-shaped members. One end of the link 422a is connected to the base unit 410 via the active joint unit 421a, the other end of the link 422a is connected to one end of the link 422b via the active joint unit 421b, and the other end of the link 422b is connected to one end of the link 422c via the active joint unit 421c. The other end of the link 422c is connected to the link 422d via a passive slide mechanism 431, and the other end of the link 422d is connected to one end of the link 422e via a passive joint unit 433. The other end of the link 422e is connected to one end of the link 422f via the active joint units 421d and 421e. The endoscope device 423 is connected to the tip of the arm unit 420, that is, to the other end of the link 422f via the active joint unit 421f.

Thus, the ends of the plurality of links 422a to 422f are connected to each other by the active joint units 421a to 421f, the passive slide mechanism 431, and the passive joint unit 433 with the base unit 410 as a fulcrum, thereby forming an arm shape extending from the base unit 410.

The actuators provided on the respective active joint units 421a to 421f of the arm unit 420 are driven and controlled to control the position and/or posture of the endoscope device 423. In the embodiment, the tip of the endoscope device 423 enters the body cavity of a patient, which is a surgical site, to image a portion of the surgical site. However, the leading end unit provided at the tip of the arm unit 420 is not limited to the endoscope device 423, and the tip of the arm unit 420 may be connected to various surgical instruments (medical tools) as leading end units. As described above, the support arm apparatus 400 according to the embodiment is configured as a medical support arm apparatus including a surgical instrument.

As illustrated in FIG. 3, the support arm apparatus 400 will be described below by defining coordinate axes. The vertical direction, the front-rear direction, and the left-right direction are defined in accordance with the coordinate axes. In other words, the vertical direction with respect to the base unit 410 installed on the floor surface is defined as the z-axis direction and the vertical direction. In addition, the directions perpendicular to the z-axis and extending the arm unit 420 from the base unit 410 (i.e., the direction in which the endoscope device 423 is positioned with respect to the base unit 410) is defined as the y-axis direction and the front-rear direction. Further, the directions perpendicular to the y-axis and the z-axis are defined as the x-axis direction and the left-right direction.

The active joint units 421a to 421f rotatably connect the links to each other. The active joint units 421a to 421f have an actuator, and a rotation mechanism driven to rotate relative to a predetermined rotation axis by driving the actuator. Controlling the rotational drive in each of the active joint units 421a to 421f allows to control the drive of the arm unit 420 such as extending or retracting (or folding) the arm unit 420. The active joint units 421a to 421f may be driven by, for example, known whole-body cooperative control and ideal joint control.

As described above, since the active joint units 421a to 421f have a rotation mechanism, in the following description, the drive control of the active joint units 421a to 421f specifically means that at least one of the rotation angle and the generated torque of the active joint units 421a to 421f is controlled. The generated torque is the torque generated by the active joint units 421a to 421f.

The passive slide mechanism 431 is an aspect of a passive form changing mechanism, and connects the link 422c and the link 422d so as to be movable forward and backward to each other along a predetermined direction. For example, the passive slide mechanism 431 may connect the link 422c and the link 422d to each other so as to be movable rectilinearly. However, the forward/backward movement of the link 422c and the link 422d is not limited to a linear movement, and may be a forward/backward movement in an arcuate direction. The passive slide mechanism 431 is operated to move forward and backward by a user, for example, to vary the distance between the active joint unit 421c on one end side of the link 422c and the passive joint unit 433. Thus, the overall form of the arm unit 420 can be changed.

The passive joint unit 433 is an aspect of a passive form changing mechanism, and rotatably connects the link 422d and the link 422e to each other. The passive joint unit 433 is rotated by a user, for example, to vary an angle formed between the link 422d and the link 422e. Thus, the overall form of the arm unit 420 can be changed.

In the present description, the “posture of the arm unit” refers to a state of an arm unit that can be changed by the drive control of an actuator provided in the active joint units 421a to 421f by a control unit in a state where the distance between adjacent active joint units across one or more links is constant.

In the present disclosure, the “posture of the arm unit” is not limited to the state of the arm unit which can be changed by the drive control of the actuator. For example, the “posture of the arm unit” may be a state of the arm unit that is changed by cooperative movement of the joint unit. Further, in the present disclosure, the arm unit need not necessarily include a joint unit. In this case, “posture of the arm unit” is a position with respect to the object or a relative angle with respect to the object.

The “form of arm unit” refers to a state of the arm unit which can be changed by changing a distance between adjacent active joint units across the link and an angle formed by the links connecting the adjacent active joint units as the passive form changing mechanism is operated.

In the present disclosure, the “form of arm unit” is not limited to the state of the arm unit which can be changed by changing the distance between adjacent active joint units across the link or the angle formed by the links connecting the adjacent active joint units. For example, the “form of arm unit” may be a state of the arm unit which can be changed by changing the positional relationship between the joint units or the angle of the joint units as the joint units are operated cooperatively. Further, in the case where the arm unit is not provided with a joint unit, the “form of arm unit” may be a state of the arm unit which can be changed by changing the position with respect to the object or the relative angle with respect to the object.

The support arm apparatus 400 illustrated in FIG. 3 includes six active joint units 421a to 421f, and six degrees of freedom are realized for driving the arm unit 420. In other words, the drive control of the support arm apparatus 400 is realized by the drive control of the six active joint units 421a to 421f by the control unit, while the passive slide mechanism 431 and the passive joint unit 433 are not subject to the drive control by the control unit.

Specifically, as illustrated in FIG. 3, the active joint units 421a, 421d, and 421f are provided so that the longitudinal axis direction of each of the connected links 422a and 422e and the imaging direction of the connected endoscope device 423 are the rotational axis direction. The active joint units 421b, 421c, and 421e are provided so that the x-axis direction, which is the direction for changing the connection angle of each of the connected links 422a to 422c, 422e, and 422f and the endoscope device 423 in the y-z plane (plane defined by the y-axis and the z-axis), is the rotational axis direction.

Thus, in the embodiment, the active joint units 421a, 421d and 421f have a function of performing so-called yawing, and the active joint units 421b, 421c and 421e have a function of performing so-called pitching.

The configuration of the arm unit 420 allows the support arm apparatus 400 applicable to the embodiment to realize six degrees of freedom for driving the arm unit 420. Therefore, the endoscope device 423 can be freely moved within the movable range of the arm unit 420. FIG. 3 illustrates a hemisphere as an example of the movable range of the endoscope device 423. Assuming that a central point RCM (remote center of motion) of the hemisphere is an imaging center of the surgical site imaged by the endoscope device 423, the surgical site can be imaged from various angles by moving the endoscope device 423 on the spherical surface of the hemisphere with the imaging center of the endoscope device 423 fixed to the central point of the hemisphere.

### 1-3. Basic Configuration of Forward-Oblique Viewing Endoscope

A basic configuration of a forward-oblique viewing endoscope will be then described as an example of an endoscope applicable to the embodiment.

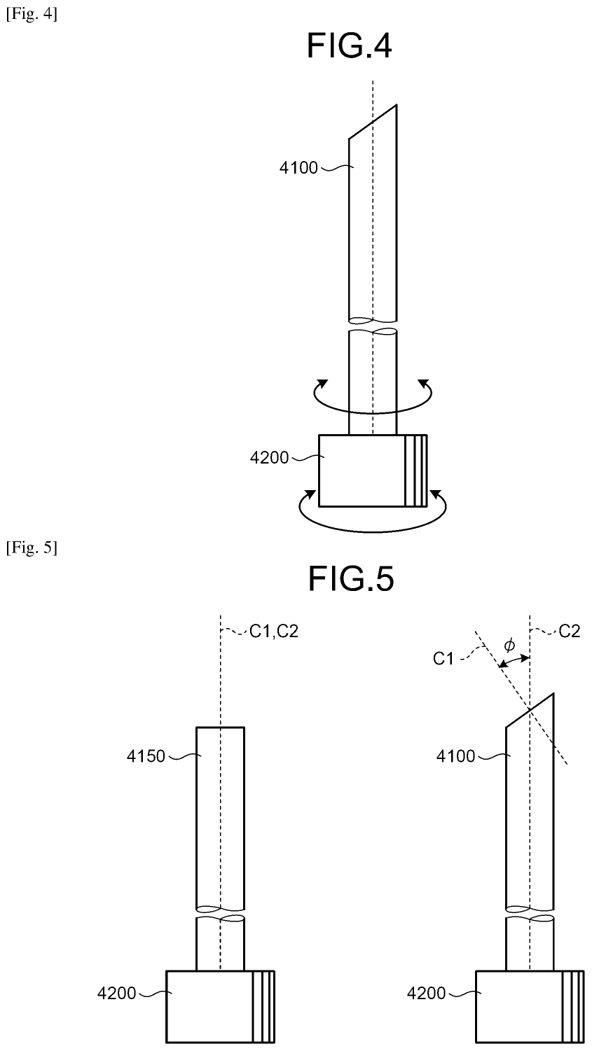

FIG. 4 is a schematic diagram illustrating a configuration of a forward-oblique viewing endoscope applicable to the embodiment. As illustrated in FIG. 4, a forward-oblique viewing endoscope 4100 is attached to a tip of a camera head 4200. The forward-oblique viewing endoscope 4100 corresponds to the lens barrel 5003 described with reference to FIGS. 1 and 2, and the camera head 4200 corresponds to the camera head 5005 described with reference to FIGS. 1 and 2.

The forward-oblique viewing endoscope 4100 and the camera head 4200 are rotatable independently of each other. An actuator (not illustrated) is provided between the forward-oblique viewing endoscope 4100 and the camera head 4200 in the same manner as the joint units 5033a, 5033b, and 5033c, and the forward-oblique viewing endoscope 4100 rotates with respect to the camera head 4200 with its longitudinal axis as a rotational axis by driving of the actuator.

The forward-oblique viewing endoscope 4100 is supported by the support arm apparatus 5027. The support arm apparatus 5027 has a function of holding the forward-oblique viewing endoscope 4100 in place of a scopist and moving the forward-oblique viewing endoscope 4100 by operation of a surgeon or an assistant so that a desired part can be observed.

FIG. 5 is a schematic diagram illustrating the forward-oblique viewing endoscope 4100 and a forward-viewing endoscope 4150 in contrast. In the forward-viewing endoscope 4150 illustrated on the left side in FIG. 5, the orientation of the objective lens to the subject (C1) coincides with the longitudinal direction of the forward-viewing endoscope 4150 (C2). On the other hand, in the forward-oblique viewing endoscope 4100 illustrated on the right side in FIG. 5, the orientation of the objective lens to the subject (C1) has a predetermined angle φ with respect to the longitudinal direction of the forward-oblique viewing endoscope 4100 (C2). The endoscope whose angle φ is 90 degrees is called a side-viewing endoscope.

### 1-4. Configuration Example of Robot Arm Apparatus Applicable to Embodiment

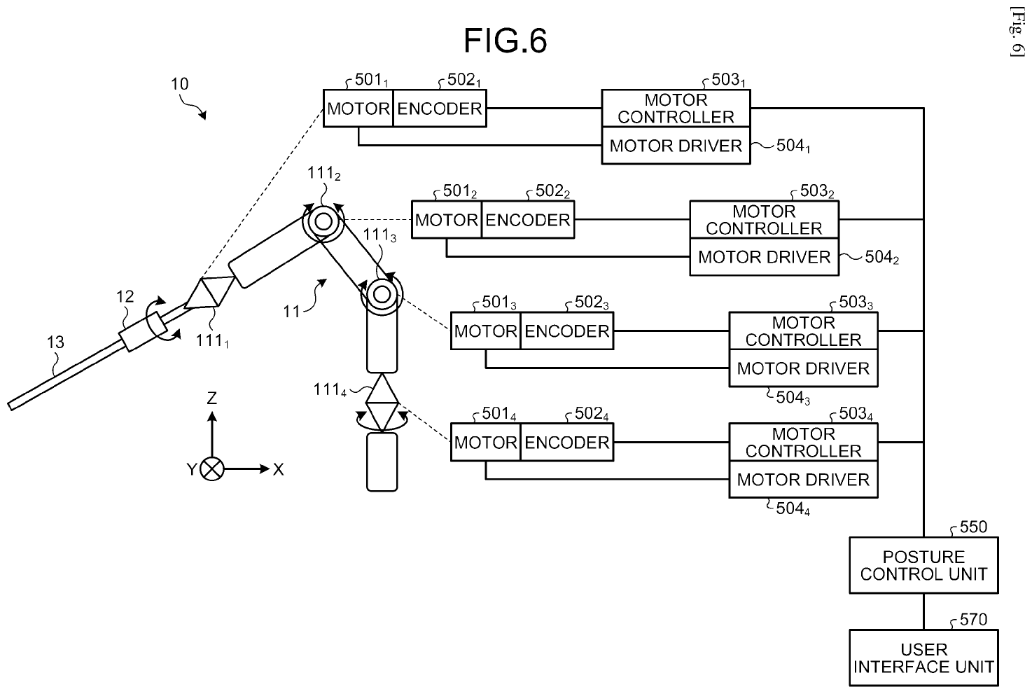

A robot arm apparatus as a support arm apparatus applicable to the embodiment will be then described more specifically. FIG. 6 is a diagram illustrating a configuration of an example of a robot arm apparatus applicable to the embodiment.

In FIG. 6, a robot arm apparatus 10 includes an arm unit 11 corresponding to the arm unit 420 in FIG. 3 and a configuration for driving the arm unit 11. The arm unit 11 includes a first joint unit 111₁, a second joint unit 111₂, a third joint unit 111₃, and a fourth joint unit 111₄. The first joint unit 111₁ supports an endoscope device 12 having a lens barrel 13. In addition, the robot arm apparatus 10 is connected to a posture control unit 550. The posture control unit 550 is connected to a user interface unit 570.

The arm unit 11 illustrated in FIG. 6 is a simplified version of the arm unit 420 described with reference to FIG. 3 for the purpose of explanation.

The first joint unit 111₁ has an actuator constituting of a motor 501₁, an encoder 502₁, a motor controller 503₁, and a motor driver 504₁.

Each of the second joint unit 111₂ to the fourth joint unit 111₄ has an actuator having the same configuration as that of the first joint unit 111₁. In other words, the second joint unit 111₂ has an actuator constituting of a motor 501₂, an encoder 502₂, a motor controller 503₂, and a motor driver 504₂. The third joint unit 111₃ has an actuator constituting of a motor 501₃, an encoder 502₃, a motor controller 503₃, and a motor driver 504₃. The fourth joint unit 111₄ also has an actuator constituting of a motor 501₄, an encoder 502₄, a motor controller 503₄, and a motor driver 504₄.

The first joint unit 111₁ to the fourth joint unit 111₄ will be described below using the first joint unit 111₁ as an example.

The motor 501₁ operates according to the control of the motor driver 504₁ and drives the first joint unit 111₁. The motor 501₁ drives the first joint unit 111₁ in both clockwise and counterclockwise directions using, for example, the direction of an arrow attached to the first joint unit 111₁, that is, the axis of the first joint unit 111₁ as a rotation axis. The motor 501₁ drives the first joint unit 111₁ to change the form of the arm unit 11 and controls the position and/or posture of the endoscope device 12.

In the example of FIG. 6, although the endoscope device 12 is provided at the base portion of the lens barrel 13, the endoscope device is not limited to this example. For example, as a form of endoscope, an endoscope device 12 may be installed at the tip of the lens barrel 13.

The encoder 502₁ detects information regarding the rotation angle of the first joint unit 111₁ according to the control of the motor controller 503₁. In other words, the encoder 502₁ acquires information regarding the posture of the first joint unit 111₁.

The posture control unit 550 changes the form of the arm unit 11 to control the position and/or posture of the endoscope device 12. Specifically, the posture control unit 550 controls the motor controllers 503₁ to 503₄, and the motor drivers 504₁ to 504₄, for example, to control the first joint unit 111₁ to the fourth joint unit 111₄. Thus, the posture control unit 550 changes the form of the arm unit 11 to control the position and/or posture of the endoscope device 12 supported by the arm unit 11. In the configuration of FIG. 1, the posture control unit 550 may be included in the arm controller 5045, for example.

The user interface unit 570 receives various operations from a user. The user interface unit 570 receives, for example, an operation for controlling the position and/or posture of the endoscope device 12 supported by the arm unit 11. The user interface unit 570 outputs an operation signal corresponding to the received operation to the posture control unit 550. In this case, the posture control unit 550 then controls the first joint unit 111₁ to the fourth joint unit 111₄ according to the operation received from the user interface unit 570 to change the form of the arm unit 11, and controls the position and/or posture of the endoscope device 12 supported by the arm unit 11.

In the robot arm apparatus 10, the captured image captured by the endoscope device 12 can be used by cutting out a predetermined region. In the robot arm apparatus 10, an electronic degree of freedom for changing a sight line by cutting out a captured image captured by the endoscope device 12 and a degree of freedom by an actuator of the arm unit 11 are all treated as degrees of freedom of a robot. Thus, motion control in which the electronic degree of freedom for changing a sight line and the degree of freedom by the actuator are linked can be realized.

### 2. Embodiment of Present Disclosure

An embodiment of the present disclosure will be then described.

### 2-1. Overview of Embodiment

An overview of the embodiment of the present disclosure will be first described. In the embodiment, the control unit that controls the robot arm apparatus 10 learns the trajectory of the position and/or posture of the endoscope device 12 in response to the operation to the position and/or posture of the endoscope device 12 by a surgeon, and generates a learned model of the position and/or posture of the endoscope device 12. The control unit predicts the position and/or posture of the endoscope device 12 at the next time by using the generated learned model, and controls the position and/or posture of the endoscope device 12 based on the prediction. Thus, the autonomous operation of the robot arm apparatus 10 is performed.

In the autonomous operation described above, there are cases in which the imaging range desired by a surgeon is not properly included in the surgical field image displayed on the display device. In this case, the surgeon evaluates that the surgical field image does not include a desired range, and gives an instruction to the robot arm apparatus 10 to stop the autonomous operation. The surgeon operates the robot arm apparatus 10 to change the position and/or posture of the endoscope device 12 so that the surgical field image captures an appropriate imaging range. When evaluating that the surgical field image includes an appropriate imaging range, the surgeon instructs the control unit to restart the autonomous operation of the robot arm apparatus 10.

When restart of the autonomous operation is instructed by the surgeon, the control unit learns the trajectory of the endoscope device 12 and corrects the learned model based on the information related to the arm unit 11 and the endoscope device 12, which is changed by changing the position and/or posture of the endoscope device 12. The control unit predicts the position and/or posture of the endoscope device 12 in the autonomous operation after restarting based on the learned model thus corrected, and drives the robot arm apparatus 10 based on the prediction.

As described above, the robot arm apparatus 10 according to the embodiment stops the autonomous operation according to the evaluation of a surgeon for the improper operation performed during the autonomous operation, corrects the learned model, and restarts the autonomous operation based on the corrected learned model. Thus, the autonomous operation of the robot arm apparatus 10 and the endoscope device 12 can be made more appropriate, and the surgical field image captured by the endoscope device 12 can be made an image including an imaging range desired by a surgeon.

### 2-2. Configuration Example of Medical Imaging System according to Embodiment

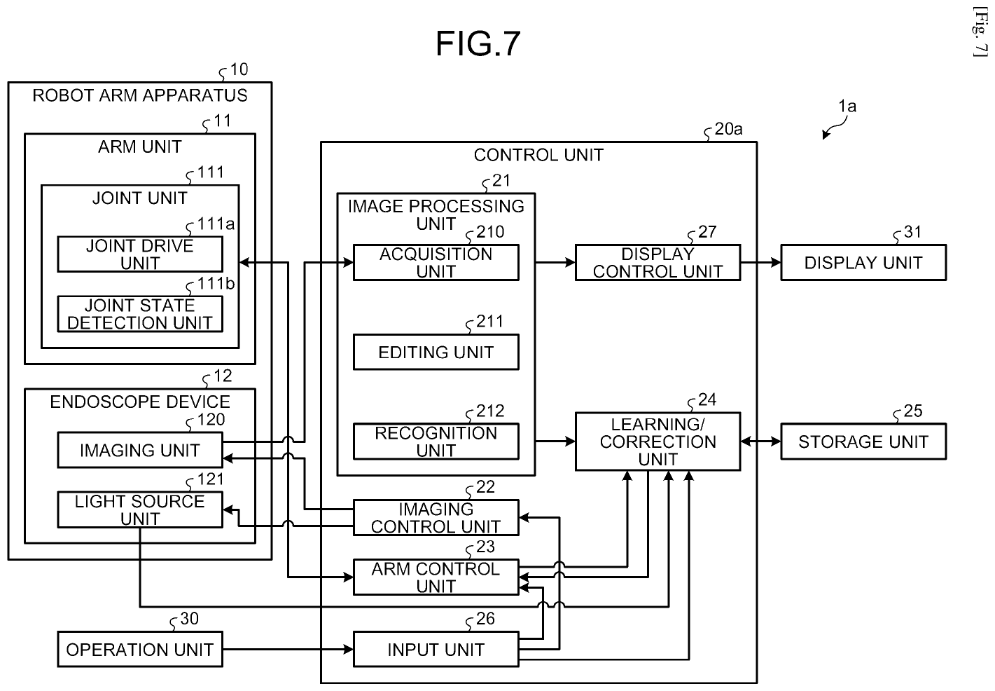

A configuration example of a medical imaging system according to the embodiment will be then described. FIG. 7 is a functional block diagram of an example for explaining a function of the medical imaging system according to the embodiment.

In FIG. 7, a medical imaging system 1a according to the embodiment includes a robot arm apparatus 10, an endoscope device 12, a control unit 20a, a storage unit 25, an operation unit 30, and a display unit 31.

Prior to the description of the configuration of the medical imaging system 1a according to the embodiment, an overview of the processing by the medical imaging system 1a will be described. In the medical imaging system 1a, first, the environment in the abdominal cavity of a patient is recognized by imaging the inside of the abdominal cavity. The medical imaging system 1a drives the robot arm apparatus 10 based on the recognition result of the environment in the abdominal cavity. Driving the robot arm apparatus 10 causes the imaging range in the abdominal cavity to change. When the imaging range in the abdominal cavity changes, the medical imaging system 1a recognizes the changed environment and drives the robot arm apparatus 10 based on the recognition result. The medical imaging system 1a repeats image recognition of the environment in the abdominal cavity and driving of the robot arm apparatus 10. In other words, the medical imaging system 1a performs processing that combines image recognition processing and processing for controlling the position and posture of the robot arm apparatus 10.

As described above, the robot arm apparatus 10 has the arm unit 11 (articulated arm) which is a multi-link structure constituted of a plurality of joint units and a plurality of links, and the arm unit 11 is driven within a movable range to control the position and/or posture of the leading end unit provided at the tip of the arm unit 11, that is, the endoscope device 12.

The robot arm apparatus 10 can be configured as the support arm apparatus 400 illustrated in FIG. 3. A description below will be given of assuming that the robot arm apparatus 10 has the configuration illustrated in FIG. 6.

Referring back to FIG. 7, the robot arm apparatus 10 includes an arm unit 11 and an endoscope device 12 supported by the arm unit 11. The arm unit 11 has a joint unit 111, and the joint unit 111 includes a joint drive unit 111a and a joint state detection unit 111b.

The joint unit 111 represents the first joint unit 111₁ to the fourth joint unit 111₄ illustrated in FIG. 6. The joint drive unit 111a is a drive mechanism in the actuator for driving the joint unit 111, and corresponds to a configuration in which the first joint unit 111₁ in FIG. 6 includes a motor 501₁ and a motor driver 504₁. The drive by the joint drive unit 111a corresponds to an operation in which the motor driver 504₁ drives the motor 501₁ with an amount of current corresponding to an instruction from an arm control unit 23 to be described below.

The joint state detection unit 111b detects the state of each joint unit 111. The state of the joint unit 111 may mean a state of motion of the joint unit 111.

For example, the information indicating the state of the joint unit 111 includes information related to the rotation of the motor such as the rotation angle, the rotation angular velocity, the rotation angular acceleration, and the generated torque of the joint unit 111. Referring to the first joint unit 111₁ in FIG. 6, the joint state detection unit 111b corresponds to the encoder 502₁. The joint state detection unit 111b may include a rotation angle detection unit that detects the rotation angle of the joint unit 111, and a torque detection unit that detects the generated torque and the external torque of the joint unit 111. In the example of the motor 501₁, the rotation angle detection unit corresponds to, for example, the encoder 502₁. In the example of the motor 501₁, the torque detection unit corresponds to a torque sensor (not illustrated). The joint state detection unit 111b transmits information indicating the detected state of the joint unit 111 to the control unit 20a.

The endoscope device 12 includes an imaging unit 120 and a light source unit 121. The imaging unit 120 is provided at the tip of the arm unit 11 and captures various imaging objects. The imaging unit 120 captures surgical field images including various surgical instruments and organs in the abdominal cavity of a patient, for example. Specifically, the imaging unit 120 includes an imaging element and a drive circuit thereof and is, for example, a camera which can image an object to be imaged in the form of a moving image or a still image. The imaging unit 120 changes the angle of view under the control of an imaging control unit 22 to be described below, and although FIG. 7 illustrates that the imaging unit 120 is included in the robot arm apparatus 10, the imaging unit is not limited to this example. In other words, the aspect of the imaging unit 120 is not limited as long as the imaging unit is supported by the arm unit 11.

The light source unit 121 irradiates an imaging object to be imaged by the imaging unit 120 with light. The light source unit 121 can be implemented by, for example, an LED for a wide-angle lens. The light source unit 121 may be configured by combining an ordinary LED and a lens, for example, to diffuse light. In addition, the light source unit 121 may be configured such that light transmitted by the optical fiber is diffused by (widen the angle of) a lens. Further, the light source unit 121 may extend the irradiation range by applying light through the optical fiber itself in a plurality of directions. Although FIG. 7 illustrates that the light source unit 121 is included in the robot arm apparatus 10, the light source unit is not limited to this example. In other words, as long as the light source unit 121 can guide the irradiation light to the imaging unit 120 supported by the arm unit 11, the aspect of the light source unit is not limited.

In FIG. 7, the control unit 20a includes an image processing unit 21, an imaging control unit 22, an arm control unit 23, a learning/correction unit 24, an input unit 26, and a display control unit 27. The image processing unit 21, the imaging control unit 22, the arm control unit 23, the learning/correction unit 24, the input unit 26, and the display control unit 27 are implemented by operating a predetermined program on the CPU. Alternatively, the image processing unit 21, the imaging control unit 22, the arm control unit 23, the learning/correction unit 24, the input unit 26, and the display control unit 27 may be partially or entirely implemented by hardware circuits operating in cooperation with each other. The control unit 20a may be included in the arm controller 5045 in FIG. 1, for example.

The image processing unit 21 performs various image processing on the captured image (surgical field image) captured by the imaging unit 120. The image processing unit 21 includes an acquisition unit 210, an editing unit 211, and a recognition unit 212.

The acquisition unit 210 acquires a captured image captured by the imaging unit 120. The editing unit 211 can process the captured image acquired by the acquisition unit 210 to generate various images. For example, the editing unit 211 can extract, from the captured image, an image (referred to as a surgical field image) relating to a display target region that is a region of interest (ROI) to a surgeon. The editing unit 211 may, for example, extract the display target region based on a determination based on a recognition result of the recognition unit 212 to be described below, or may extract the display target region in response to an operation of the operation unit 30 by a surgeon. Further, the editing unit 211 can extract the display target region based on the learned model generated by the learning/correction unit 24 to be described below.

For example, the editing unit 211 generates a surgical field image by cutting out and enlarging a display target region of the captured image. In this case, the editing unit 211 may be configured to change the cutting position according to the position and/or posture of the endoscope device 12 supported by the arm unit 11. For example, when the position and/or posture of the endoscope device 12 is changed, the editing unit 211 can change the cutting position so that the surgical field image displayed on the display screen of the display unit 31 does not change.

Further, the editing unit 211 performs various image processing on the surgical field image. The editing unit 211 can, for example, perform high-quality image processing on the surgical field image. The editing unit 211 may, for example, perform super-resolution processing on the surgical field image as high-quality image processing. The editing unit 211 may also perform, for example, band enhancement processing, noise reduction processing, camera shake correction processing, and luminance correction processing, as high-quality image processing, on the surgical field image. In the present disclosure, the high-quality image processing is not limited to these processing, but may include various other processing.

Further, the editing unit 211 may perform low resolution processing on the surgical field image to reduce the capacity of the surgical field image. In addition, the editing unit 211 can perform, for example, distortion correction on the surgical field image. Applying distortion correction on the surgical field image allows the recognition accuracy by the recognition unit 212 which will be described below to be improved.

The editing unit 211 can also change the type of image processing such as correction on the surgical field image according to the position where the surgical field image is cut from the captured image. For example, the editing unit 211 may correct the surgical field image by increasing the intensity toward the edge stronger than the central region of the surgical field image. Further, the editing unit 211 may or may not correct the central region of the surgical field image by decreasing the intensity. Thus, the editing unit 211 can perform optimum correction on the surgical field image according to the cutting position. Therefore, the recognition accuracy of the surgical field image by the recognition unit 212 can be improved. In general, since the distortion of a wide-angle image tends to increase toward the edge of the image, a surgical field image that enables a surgeon to grasp the state of the surgical field without feeling uncomfortable can be generated by changing the intensity of correction according to the cutting position.

Further, the editing unit 211 may change the processing to be performed on the surgical field image based on the information inputted to the control unit 20a. For example, the editing unit 211 may change the image processing to be performed on the surgical field image, based on at least one of the information on the movement of each joint unit 111 of the arm unit 11, the recognition result of the surgical field environment based on the captured image, and the object and treatment status included in the captured image. The editing unit 211 changes the image processing according to various situations, so that a surgeon, for example, can easily recognize the surgical field image.

The recognition unit 212 recognizes various pieces of information, for example, based on the captured image acquired by the acquisition unit 210. The recognition unit 212 can recognize, for example, various types of information regarding surgical instruments (surgical tools) included in the captured image. For example, the recognition unit 212 can recognize various types of information regarding organs included in the captured image.

The recognition unit 212 can recognize the types of various surgical instruments included in the captured image based on the captured image. In the recognition, the imaging unit 120 includes a stereo sensor, and the type of the surgical instrument can be recognized with higher accuracy by using a captured image captured by using the stereo sensor. The types of surgical instruments recognized by the recognition unit 212 include, but are not limited to, forceps, scalpels, retractors, and endoscopes, for example.

Further, the recognition unit 212 can recognize, based on the captured image, the coordinates of various surgical instruments included in the captured image in the abdominal cavity in the three-dimensional orthogonal coordinate system. More specifically, the recognition unit 212 recognizes, for example, the coordinates (x₁, y₁, z₁) of one end and the coordinates (x₂, y₂, z₂) of the other end of the first surgical instrument included in the captured image. The recognition unit 212 recognizes, for example, the coordinates (x₃, y₃, z₃) of one end and the coordinates (x₄, y₄, z₄) of the other end of the second surgical instrument included in the captured image.

Further, the recognition unit 212 can recognize the depth in the captured image. For example, the imaging unit 120 includes a depth sensor, and the recognition unit 212 can measure the depth based on the image data measured by the depth sensor. Thus, the depth of the body included in the captured image can be measured, and the three-dimensional shape of the organ can be recognized by measuring the depth of a plurality of body parts.

Further, the recognition unit 212 can recognize the movement of each surgical instrument included in the captured image. For example, the recognition unit 212 recognizes, for example, the motion vector of the image of the surgical instrument recognized in the captured image, thereby recognizing the movement of the surgical instrument. The motion vector of the surgical instrument can be acquired using, for example, a motion sensor. Alternatively, a motion vector may be obtained by comparing captured images captured as moving images between frames.

Further, the recognition unit 212 can recognize the movement of the organs included in the captured image. The recognition unit 212 recognizes, for example, the motion vector of the image of the organ recognized in the captured image, thereby recognizing the movement of the organ. The motion vector of the organ can be acquired using, for example, a motion sensor. Alternatively, a motion vector may be obtained by comparing captured images captured as moving images between frames. Alternatively, the recognition unit 212 may recognize the motion vector by an algorithm related to image processing such as optical flow based on the captured image. Processing for canceling the movement of the imaging unit 120 may be executed based on the recognized motion vector.

Thus, the recognition unit 212 recognizes at least one of objects, such as a surgical instrument and an organ, and a treatment status, including the movement of the surgical instrument.

The imaging control unit 22 controls the imaging unit 120. For example, the imaging control unit 22 controls the imaging unit 120 to image the surgical field. For example, the imaging control unit 22 controls the magnification ratio of imaging by the imaging unit 120. The imaging control unit 22 controls the imaging operation including the change of the magnification ratio of the imaging unit 120 in response to, for example, the operation information from the operation unit 30 inputted to the input unit 26 to be described below and instructions from the learning/correction unit 24 to be described below.

The imaging control unit 22 further controls the light source unit 121. The imaging control unit 22 controls the brightness of the light source unit 121 when the imaging unit 120 images the surgical field, for example. The imaging control unit 22 can control the brightness of the light source unit 121 in response to an instruction from the learning/correction unit 24, for example. The imaging control unit 22 can also control the brightness of the light source unit 121 based on, for example, the positional relationship of the imaging unit 120 with respect to the region of interest. Further, the imaging control unit 22 can control the brightness of the light source unit 121 in response to, for example, the operation information from the operation unit 30 inputted to the input unit 26.

The arm control unit 23 integrally controls the robot arm apparatus 10 and controls the drive of the arm unit 11. Specifically, the arm control unit 23 controls the drive of the joint unit 111 to control the drive of the arm unit 11. More specifically, the arm control unit 23 controls the number of rotations of the motor by controlling the amount of current supplied to the motor (for example, the motor 501₁) in the actuator of the joint unit 111, and controls the rotation angle and the generated torque in the joint unit 111. Thus, the arm control unit 23 can control the form of the arm unit 11 and control the position and/or posture of the endoscope device 12 supported by the arm unit 11.

The arm control unit 23 can control the form of the arm unit 11 based on the determination result for the recognition result of the recognition unit 212, for example. The arm control unit 23 controls the form of the arm unit 11 based on the operation information from the operation unit 30 inputted to the input unit 26. Further, the arm control unit 23 can control the form of the arm unit 11 in response to an instruction based on the learned model by the learning/correction unit 24 to be described below.

The operation unit 30 has one or more operation elements and outputs operation information according to the operation with respect to the operation elements by a user (for example, a surgeon). As the operation elements of the operation unit 30, a switch, a lever (including a joystick), a foot switch, and a touch panel, for example, which are operated by the user directly or indirectly in contact with each other can be applied. Alternatively, a microphone for detecting voice or a sight line sensor for detecting a sight line can be applied as an operation element.

The input unit 26 receives various types of operation information outputted by the operation unit 30 in response to a user operation. The operation information may be inputted by a physical mechanism (for example, an operation element) or by voice (voice input will be described below). The operation information from the operation unit 30 is, for example, instruction information for changing the magnification ratio (zoom amount) of the imaging unit 120 and the position and/or posture of the arm unit 11. The input unit 26 outputs, for example, instruction information to the imaging control unit 22 and the arm control unit 23. The imaging control unit 22 controls the magnification ratio of the imaging unit 120 based on, for example, instruction information received from the input unit 26. The arm control unit 23 controls the position/posture of the arm unit 11 based on, for example, instruction information received from a reception unit.

Further, the input unit 26 outputs a trigger signal to the learning/correction unit 24 in response to a predetermined operation to the operation unit 30.

The display control unit 27 generates a display signal that can be displayed by the display unit 31 based on the surgical field image or the captured image outputted from the image processing unit 21. The display signal generated by the display control unit 27 is supplied to the display unit 31. The display unit 31 includes a display device such as a liquid crystal display (LCD) or an organic electro-luminescence (EL) display, and a drive circuit for driving the display device. The display unit 31 displays an image or video on the display region of the display device according to the display signal supplied from the display control unit 27. The surgeon can perform the endoscopic surgery while viewing the images and videos displayed on the display unit 31.

The storage unit 25 stores data in a nonvolatile state and reads out the stored data. The storage unit 25 may be a storage device including a nonvolatile storage medium such as a hard disk drive or a flash memory, and a controller for writing data to and reading data from the storage medium.

The learning/correction unit 24 learns, as learning data, various types of information acquired from the robot arm apparatus 10 and input information inputted to the input unit 26 including operation information in response to the operation to the operation unit 30, and generates a learned model for controlling the drive of each joint unit 111 of the robot arm apparatus 10. The learning/correction unit 24 generates an arm control signal for controlling the drive of the arm unit 11 based on the learned model. The arm unit 11 can execute autonomous operation according to the arm control signal.

Further, the learning/correction unit 24 corrects the learned model according to a trigger signal outputted from the input unit 26 in response to, for example, an operation to the operation unit 30, and overwrites the learned model before correction with the corrected learned model.

The learning/correction unit 24 then outputs an arm control signal for stopping the autonomous operation of the arm unit 11 in response to the trigger signal received from the input unit 26. The arm unit 11 stops the autonomous operation based on the learned model in response to the arm control signal. While the autonomous operation of the arm unit 11 is closely observed, the position and/or posture of the endoscope device 12 can be manually corrected.

Further, the learning/correction unit 24 outputs an arm control signal for restarting the drive control of the arm unit 11 in response to a trigger signal received from the input unit 26 following the trigger signal. In response to the arm control signal, the arm unit 11 restarts autonomous operation using the corrected learned model.

A trigger signal for stopping the autonomous operation of the arm unit 11 and starting a correction operation is hereinafter referred to as a start trigger signal. A trigger signal for terminating the correction operation and restarting the autonomous operation is also referred to as an end trigger signal.

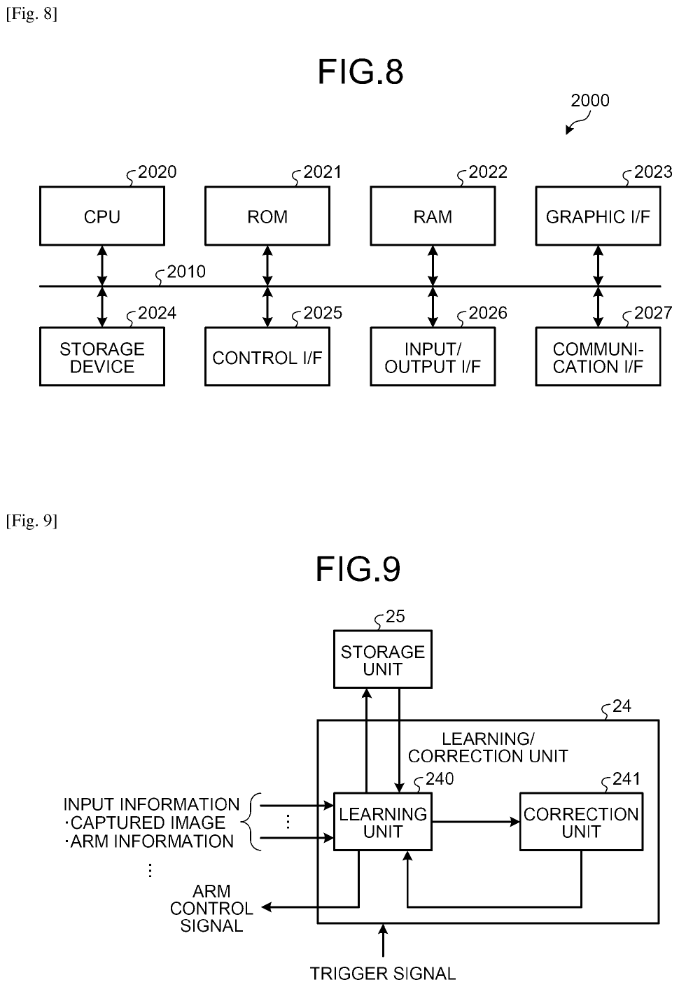

FIG. 8 is a block diagram illustrating a configuration of an example of a computer capable of implementing the control unit 20a according to the embodiment. For example, a computer 2000 is mounted on the cart 5037 illustrated in FIG. 1 to implement the function of the arm controller 5045. The function of the control unit 20a may be included in the arm controller 5045.

The computer 2000 includes a CPU 2020, a read only memory (ROM) 2021, a random access memory (RAM) 2022, a graphic I/F 2023, a storage device 2024, a control I/F 2025, an input/output I/F 2026, and a communication I/F 2027, and the respective components are connected to each other by a bus 2010 so as to be communicable.

The storage device 2024 includes a nonvolatile storage medium such as a hard disk drive or a flash memory, and a controller for writing and reading data on the storage medium.

The CPU 2020, in accordance with programs stored in the storage device 2024 and the ROM 2021, uses the RAM 2022 as a work memory to control the overall operation of the computer 2000. The graphic I/F 2023 converts the display control signal generated by the CPU 2020 in accordance with the program into a display signal in a format displayable by the display device.

The control I/F 2025 is an interface to the robot arm apparatus 10. The CPU 2020 communicates via the control I/F 2025 with the arm unit 11 and the endoscope device 12 of the robot arm apparatus 10 to control the operation of the arm unit 11 and the endoscope device 12. The control I/F 2025 can also connect various recorders and measuring devices.

The input/output I/F 2026 is an interface to an input device and an output device connected to the computer 2000. Input devices connected to the computer 2000 include a pointing device such as a mouse or a touch pad, and a keyboard. Alternatively, various switches, levers, and joysticks, for example, can be applied as input devices. Examples of the output device connected to the computer 2000 include a printer and a plotter. A speaker can also be applied as an output device.

Further, the captured image captured by the imaging unit 120 in the endoscope device 12 can be inputted via the input/output I/F 2026 to the computer 2000. The captured image may be inputted via the control I/F 2025 to the computer 2000.

The communication I/F 2027 is an interface for performing communication with an external device by wire or wireless. The communication I/F 2027 can be connected to a network such as a local area network (LAN), for example, and can communicate with network devices such as a server device and a network printer via the network, or can communicate with the Internet.

For example, the CPU 2020 constitutes the image processing unit 21, the imaging control unit 22, the arm control unit 23, the learning/correction unit 24, the input unit 26, and the display control unit 27 described above on the main storage area of the RAM 2012 as modules, for example, by executing the program according to the embodiment. The modules constituting the learning/correction unit 24 are configured on the main storage area, for example, by executing the learned model generation program included in the program by the CPU 2020.

The program can be acquired, for example, by communication through the communication I/F 2027 from an external (for example, a server device) and installed on the computer 2000. Alternatively, the program may be stored in a removable storage medium such as a compact disk (CD), a digital versatile disk (DVD), or a universal serial bus (USB) memory. The learned model generation program may be provided and installed separately from the program.

### 2-3. Overview of Processing by Medical Imaging System according to Embodiment

An overview of processing by a medical imaging system according to the embodiment will be then described. A description below will be given of a medical imaging system 1b corresponding to the operation to the operation unit 30 and the voice input described with reference to FIG. 9.

FIG. 9 is a functional block diagram of an example for explaining a function of the learning/correction unit 24 according to the embodiment. In FIG. 8, the learning/correction unit 24 includes a learning unit 240 and a correction unit 241.

The learning unit 240 learns at least one of the trajectory of a surgical instrument (for example, forceps) and the trajectory of the endoscope device 12 from, for example, a data sample based on an actual operation by a surgeon to generate a learned model, and performs prediction based on the learned model. The learning unit 240 generates an arm control signal based on the prediction to drive and control the arm unit 11, and makes the trajectory of the endoscope device 12 follow the prediction based on the learned model.

The surgeon actually uses the arm unit 11 driven and controlled according to the prediction based on the learned model and the endoscope device 12 supported by the arm unit 11, and generates evaluation during use. The occurrence of the evaluation is notified by a trigger signal (a start trigger signal and an end trigger signal) outputted from the input unit 26 to the learning/correction unit 24.

The correction unit 241 provides an interface for relearning the learned model by using information indicating the trajectory of the endoscope device 12 at the time of occurrence of evaluation. In other words, the correction unit 241 acquires a correct answer label according to the evaluation by the surgeon, relearns the learned model based on the correct answer label, and realizes an interface for correcting the learned model.

The evaluation occurs, for example, when an abnormality or a sense of incongruity is found in the surgical field image captured by the endoscope device 12 and the autonomous operation of the arm unit 11 is stopped by the surgeon, and when the position and/or posture of the endoscope device 12 is corrected by the surgeon so that the abnormality or sense of incongruity in the surgical field image is eliminated. In the evaluation, the correct answer label at the time when the autonomous operation of the arm unit 11 is stopped by the surgeon is a value indicating an incorrect answer (for example, “0”), and the correct answer label at the time when the position and/or posture of the endoscope device 12 is corrected is a value indicating a correct answer (for example, “1”).

### 2-4. Details of Processing by Medical Imaging System according to Embodiment

The processing by the medical imaging system according to the embodiment will be then described in more detail. In the embodiment, the position and/or posture of the endoscope device 12, for example, the position of the tip (tip of the lens barrel 13) of the endoscope device 12, is controlled based on the position of the surgical instrument used by the surgeon.

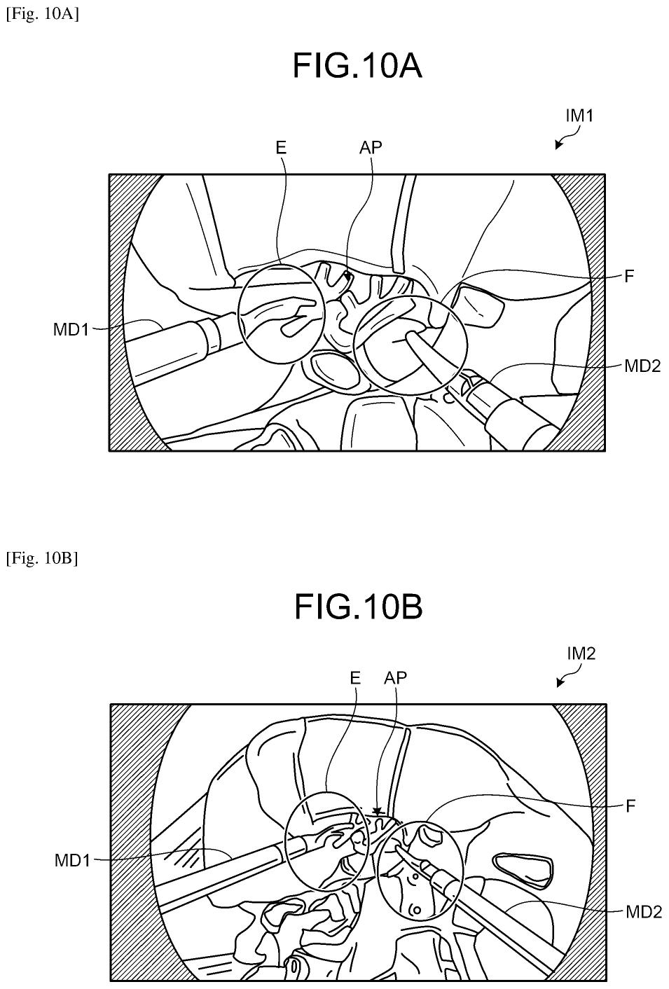

FIGS. 10A and 10B are diagrams illustrating examples of captured images captured by the endoscope device 12. A captured image IM1 illustrated in FIG. 10A and a captured image IM2 illustrated in FIG. 10B are images obtained by imaging a range including the same surgical field at different magnification ratios, and the captured image IM1 has a larger magnification ratio (zoom amount) than the captured image IM2. Taking FIG. 10A as an example, the captured image IM includes images of surgical instruments MD1 and MD2 operated by a surgeon and an image of a surgical target site AP. In FIG. 10A, the leading end of the surgical instrument MD1 is illustrated at position E, and the leading end of the MD2 is illustrated at position F. The positions E and F of the leading ends of the surgical instruments MD1 and MD2 are hereinafter set as the positions of the surgical instruments MD1 and MD2, respectively.

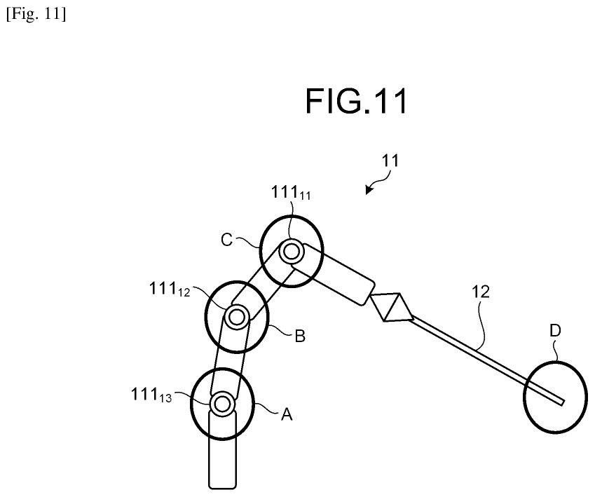

FIG. 11 is a schematic diagram for explaining the control of the arm unit 11 according to the embodiment. In the example of FIG. 11, the arm unit 11 includes, as movable portions, a first joint unit 111₁₁, a second joint unit 111₁₂, and a third joint unit 111₁₃ illustrated as A, B, and C in the figure. The support portion connected to the first joint unit 111₁₁ supports the endoscope device 12. In FIG. 11, the endoscope device 12 is represented by a lens barrel.

In the embodiment, based on the positions of the surgical instruments MD1 and MD2 described with reference to FIGS. 10A and 10B, the position and/or posture of the leading end (illustrated as D in FIG. 11) of the endoscope device 12 supported by the arm unit 11 is controlled.

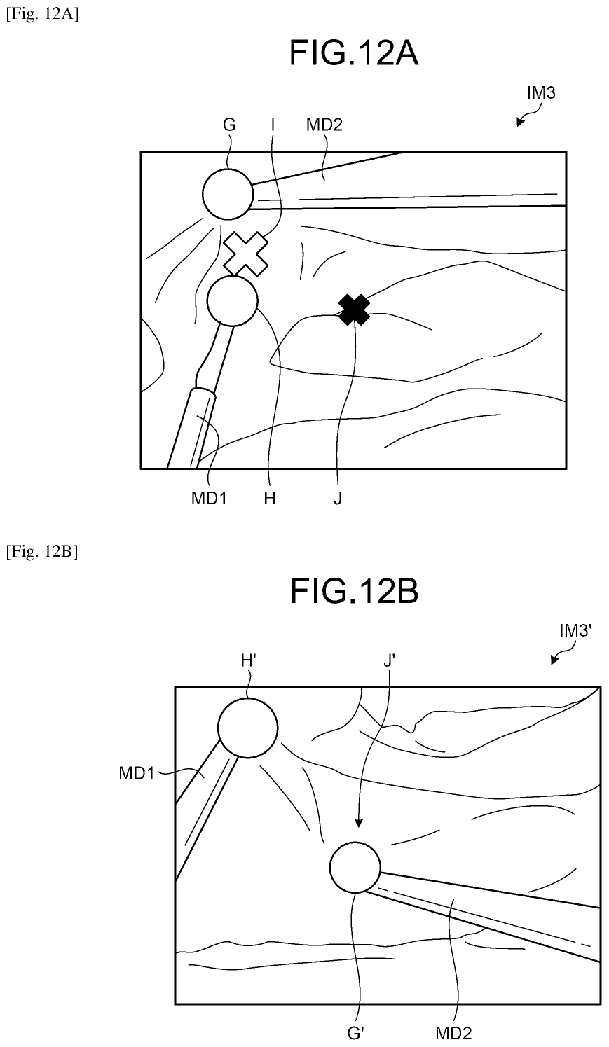

FIGS. 12A and 12B are schematic diagrams for schematically explaining processing by the learning unit 240 according to the embodiment.

FIG. 12A illustrates an example of a gaze point assumed by the existing technique. In a captured image IM3, the surgical instruments MD1 and MD2 are placed on positions H and G, respectively, and a surgeon's gaze point is assumed to be a position I of a substantially intermediate point between the positions H and G. Therefore, in the existing technique, for example, the position and/or posture of the endoscope device 12 has been controlled so that the position I is located substantially at the center of the captured image IM3.

For example, when the actual gaze point of the surgeon is a position J at a position apart from the position I, and the position and/or posture of the endoscope device 12 is controlled so that the position I is located at the center of the captured image IM3, the position J, which is the actual gaze point, moves to the peripheral portion of the captured image IM3, and a preferable surgical field image for the surgeon cannot be obtained. Therefore, the position I is an inappropriate prediction position.

FIG. 12B illustrates an example in which the learning unit 240 according to the embodiment properly predicts the gaze point of the surgeon with respect to the captured image IM3 in FIG. 12A. In the example of FIG. 12B, in a captured image IM3′, the position and/or posture of the endoscope device 12 is controlled so that a position J′ corresponding to the position J in FIG. 12A is substantially centered, and the surgical instrument MD2 is placed at the position J′ (a position G′). Further, the surgical instrument MD1 is moved to a position H′ corresponding to the position G in FIG. 12A. Thus, predicting the actual gaze point of the surgeon using the learned model learned by the learning unit 240 according to the embodiment and controlling the position and/or posture of the endoscope device 12 according to the predicted gaze point allows the surgeon to easily perform the surgery.

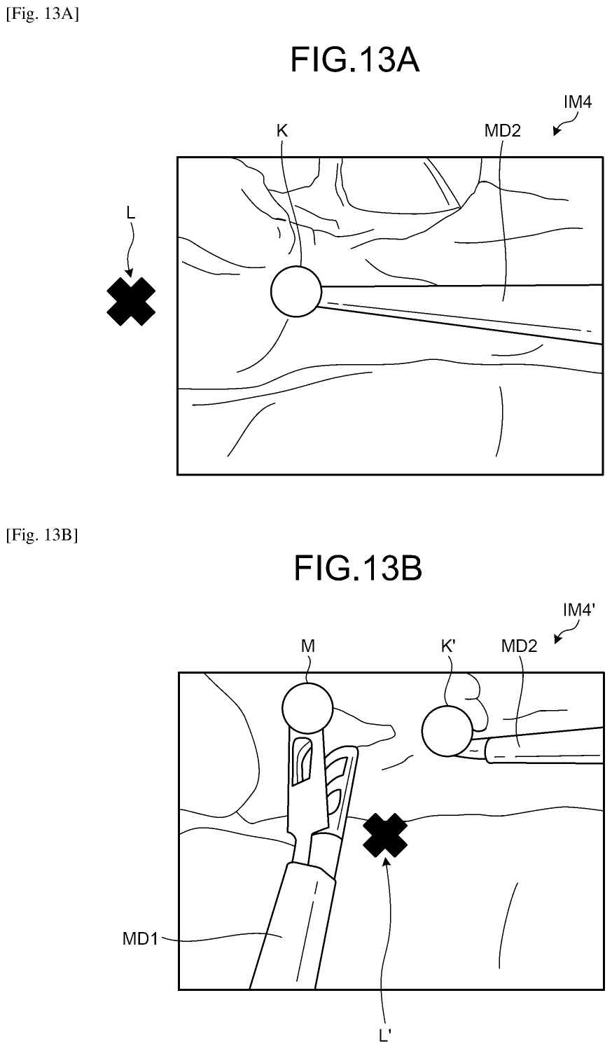

FIGS. 13A and 13B are schematic diagrams for schematically explaining processing by the correction unit 241 according to the embodiment.

FIG. 13A illustrates an example of a captured image IM4 captured by the predicted improper position and/or posture of the endoscope device 12. In the example of FIG. 13A, only the surgical instrument MD2 of the surgical instruments MD1 and MD2 used by the surgeon, which is placed at a position K, is included in the image. In the image, the assumption is made that the actual surgeon's gaze point is a position L protruding from the captured image IM4.

In the example of FIG. 13A, the captured image IM4 does not include the gaze point desired by the surgeon and does not include, for example, the other surgical instrument MD1, which may interfere with the surgeon's treatment. Therefore, the surgeon manually corrects the position and/or posture of the endoscope device 12 by stopping the autonomous operation of the robot arm apparatus 10 by, for example, the operation to the operation unit 30 or by voice.

FIG. 13B illustrates an example of a captured image IM4′ captured by the endoscope device 12 whose position and/or posture has been corrected by a surgeon. In the captured image IM4′, a position L′ of a gaze point desired by the surgeon is located substantially at the center of the captured image IM4′, and the surgical instruments MD1 and MD2 used by the surgeon are included in the captured image IM4′. The correction unit 241 corrects the learned model generated by the learning unit 240 by using the position and/or posture of the endoscope device 12 thus corrected and positions M and K′ of the respective surgical instruments MD1 and MD2.

Predicting and controlling the position and/or posture of the endoscope device 12 by the corrected learned model makes the imaging range of the captured image captured by the endoscope device 12 appropriate, enabling the autonomous operation of the endoscope device 12 and the arm unit 11 supporting the endoscope device 12.

### 2-4-1. Processing of Learning Unit according to Embodiment

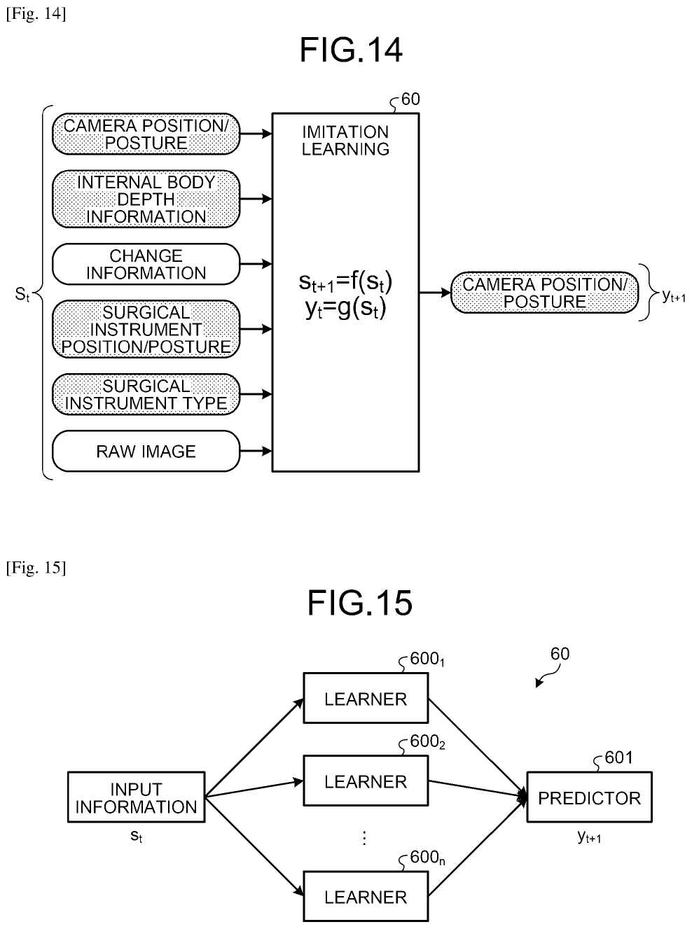

The processing in the learning unit 240 according to the embodiment will be described. FIG. 14 is a schematic diagram for explaining the learning processing in the learning unit 240 according to the embodiment. The learning unit 240 uses a learning model 60 to perform imitation learning using a plurality of pieces of input information sₜ at time t, and outputs output information yₜ₊₁ as a predicted value at the next time t+1. In the embodiment, the learning unit 240 measures surgery data related to the surgery by the surgeon, and learns the learning model 60 using the trajectory of the surgery data.

More specifically, the learning unit 240 uses the position and/or posture of the surgical instrument such as forceps used by the surgeon in the surgery and the position and/or posture of the endoscope device 12 (arm unit 11) during the surgery when the surgeon's assistant (another surgeon, a scopist, or the like) manually moves the endoscope device 12 (arm unit 11) to learn the learning model 60.

A data set for learning the first learning model 60 is generated in advance. The data set may be generated by actually measuring the surgery performed by a plurality of surgeons or by simulation. The medical imaging system 1a stores the data set in advance, for example, in the storage unit 25. Alternatively, the data set may be stored in a server on the network.

The position and/or posture of the surgical instrument used by the surgeon, and the position and/or posture of the endoscope device 12 when the surgeon's assistant moves the endoscope device 12, can be measured using a measuring device such as, for example, motion capture.

Alternatively, the position and/or posture of the surgical instrument used by the surgeon can be detected based on the captured image captured by the endoscope device 12. In this case, for example, the position and/or posture of the surgical instrument can be detected by comparing the results of the recognition processing by the recognition unit 212 for the captured image in a plurality of frames. Further, when a surgeon's assistant manually moves the robot arm apparatus 10 by an operation with respect to an operation element arranged in the operation unit 30, the state of each joint unit 111 of the arm unit 11 can be known based on information such as an encoder, which can be used to measure the position and/or posture of the endoscope device 12. In addition to the position and/or posture of the endoscope device 12, the posture of the endoscope device 12 is preferably measured.

The input information sₜ includes, for example, the current (time t) position and/or posture of the endoscope device 12 and the position and/or posture of the surgical instrument. Further, the output information yₜ₊₁ includes, for example, the position and/or posture of the endoscope device 12 at the next time (time t+1) used for control. In other words, the output information yₜ₊₁ is a predicted value obtained by predicting, at time t, the position and/or posture of the endoscope device 12 at time t+1.

The input information sₜ is not limited to the current position and/or posture of the endoscope device 12 and the position and/or posture of the surgical instrument. In the example of FIG. 14, as the input information sₜ, camera position/posture, internal body depth information, change information, surgical instrument position/posture, surgical instrument type, and RAW image are provided, and the camera position/posture, internal body depth information, surgical instrument position/posture, and surgical instrument type are used for learning the learning model 60. For example, the learning unit 240 sequentially tries to learn the learning model 60 from the minimum set based on each of the available input information sₜ.

In the input information sₜ, “camera position/posture” is the position and/or posture of the endoscope device 12. The “internal body depth information” is information indicating the depth in the range of the captured image in the abdominal cavity measured by the recognition unit 212 using the depth sensor. The “change information” is, for example, information indicating a change in the surgical target site AP. The “surgical instrument position/posture” is information indicating the position and/or posture of the surgical instrument included in the captured image. The “surgical instrument type” is information indicating the type of the surgical instrument included in the captured image. The RAW image is captured by the endoscope device 12 and is not subjected to demosaic processing. The “change information”, “surgical instrument position/posture”, and “surgical instrument type” can be acquired, for example, based on the recognition processing for the captured image by the recognition unit 212.

The input information sₜ illustrated in FIG. 14 is an example, and is not limited thereto.

The learning model 60 predicts the position and/or posture of the endoscope device 12 at the next time by the following equations (1) and (2).

sₜ₊₁=f(sₜ) (1)

yₜ=g(sₜ) (2)

The equation (1) illustrates that the input information sₜ₊₁ at time t+1 is represented by a function f of the input information sₜ at time t. Further, the equation (2) illustrates that the output information yₜ at time t is represented by a function g of the input information sₜ at time t. Combining these equations (1) and (2) allows output information yₜ₊₁ at time t+1, which is the next time, to be predicted at time t.

The learning unit 240 learns, in the learning model 60, the functions f and g based on each of the input information sₜ and output information yₜ. These functions f and g change sequentially. The functions f and g are also different depending on surgeons.

FIG. 15 is a schematic diagram for explaining an example of the learning model 60 according to the embodiment. The learning model 60 according to the embodiment can be generated by ensemble learning using a plurality of learners (prediction model). In the example of FIG. 15, the learning model 60 includes a plurality of learners 600₁, 600₂, . . . , 600ₙ. Each of the learners 600₁, 600₂, . . . , 600ₙ can apply a weak learner.

The input information sₜ is inputted to each of the learners 600₁, 600₂, . . . , 600ₙ. The outputs of each of the learners 600₁, 600₂, . . . , 600ₙ are inputted to a predictor 601. The predictor 601 integrates each of the learners 600₁, 600₂, . . . , 600ₙ to obtain output information yₜ₊₁ which is a final predicted value. When determined that the learning by the learning model 60 has been sufficiently performed, the learning unit 240 stores the learned learning model 60 as a learned learning model, for example, in the storage unit 25.

Using ensemble learning allows highly accurate output information yₜ₊₁ from relatively little input information sₜ to be obtained.

The learning method of the learning model 60 is not particularly limited as long as the method is a learning method using a nonlinear model. At the time of consideration of the present disclosure, the applicants of the present disclosure have learned nonlinear functions using the Gaussian process (GP) which is a nonlinear model with a small amount of data. Since the learning method depends on the learning data, GP can be replaced by another nonlinear function learning method. As another example of the nonlinear function learning method, a stochastic model including dynamics such as a mixed Gaussian model (GMM), a Kalman filter (KF), a hidden Markov model (HMM), and a method using SQL Server Management Studio (SSMS) can be considered. Alternatively, deep learning methods such as convolutional neural network (CNN) and recurrent neural network (RNN) can also be applied.

In the above description, although the learning model 60 is based on boosting as an ensemble learning method, the learning model is not limited to this example. For example, the learning/correction unit 24 may learn the learning model 60 by using, as an ensemble learning method, a random forest in which a decision tree is used as a weak learner, and bagging in which diversity is given to a data set by restoring and extracting learning data, for example.

The data set for learning the first learning model 60 may be stored locally in the medical imaging system 1a, or may be stored, for example, in a cloud network.

Generally, the pattern of the surgery is different for each surgeon, and accordingly, the trajectory of the endoscope device 12 is also different for each surgeon. Therefore, the learning/correction unit 24 performs learning such as the trajectory of the endoscope device 12 for each surgeon, generates a learned model for each surgeon, and stores the generated learned model in the storage unit 25 in association with information identifying the surgeon, for example. The learning/correction unit 24 reads the learned model corresponding to the surgeon from the learned model stored in the storage unit 25 and applies the learned model, according to the authentication information of the surgeon to the medical imaging system 1a and the selection from the list of surgeons presented from the medical imaging system 1a.

### 2-4-2. Processing of Correction Unit according to Embodiment

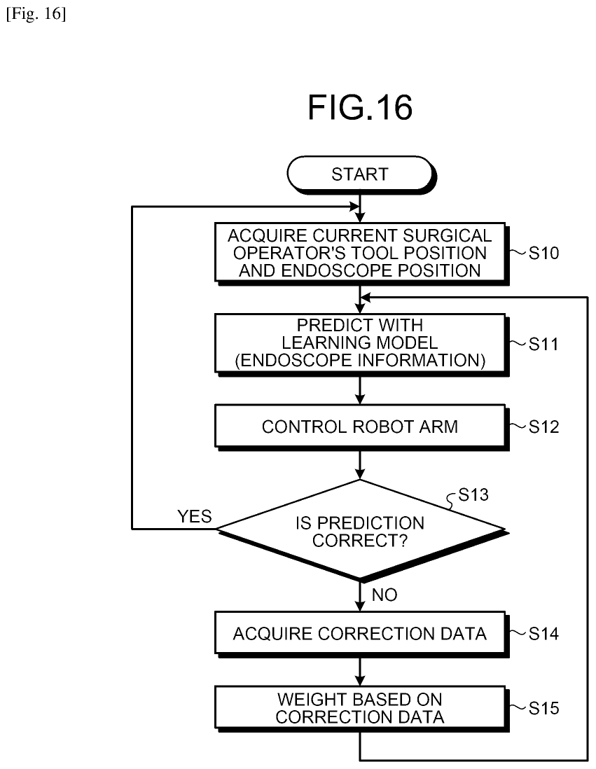

The processing in the correction unit 241 according to the embodiment will be described. FIG. 16 is a flowchart illustrating an example of processing by the learning/correction unit 24 according to the embodiment.

For the purpose of explanation, the assumption is made that the input information Sₜ to the learning unit 240 is the position of the endoscope device 12 and the position of the surgical instrument used by a surgeon, and the output information yₜ₊₁ is the position of the endoscope device 12. Further, the assumption is made that the operation mode of the robot arm apparatus 10 is an autonomous operation mode in which autonomous operation based on a previously generated learned model is performed at the initial stage of the flowchart.

In step S10, the learning/correction unit 24 acquires the position of the tool (surgical instrument) of the current (time t) surgeon and the position of the endoscope device 12. The position of the surgical instrument can be acquired based on the result of the recognition processing of the surgical instrument with respect to the captured image by the recognition unit 212. The position of the endoscope device 12 can be acquired from the arm control unit 23.

In the next step S11, the learning/correction unit 24 uses the learning unit 240 to predict, based on the position of the surgical instrument and the endoscope device 12 at time t acquired in the step S10, the position of the endoscope device 12 at the next time t+1 according to the learned model. The learning unit 240 holds information indicating the predicted position of the endoscope device 12 as endoscope information, for example.

In the next step S12, the learning/correction unit 24 uses the learning unit 240 to perform the robot arm control processing based on the endoscope information held in the step S11. More specifically, the learning unit 240 generates an arm control signal based on the endoscope information held in the step S11, and passes the generated arm control signal to the arm unit 11. The arm unit 11 drives and controls each joint unit 111 according to the arm control signal passed. Thus, the robot arm apparatus 10 is autonomously controlled.

In the next step S13, the learning/correction unit 24 determines whether the prediction in the step S11 is correct. More specifically, in the case where the start trigger signal is outputted from the input unit 26, the learning/correction unit 24 determines that the prediction is not correct (an incorrect answer).

For example, when the captured image (surgical field image) displayed on the display unit 31 is captured in an abnormal or unnatural imaging range as illustrated in FIG. 13A, the surgeon instructs the operation unit 30 to stop the autonomous operation by the robot arm apparatus 10. The input unit 26 outputs a start trigger signal to the learning/correction unit 24 in response to the operation to the operation unit 30.

If determined in the step S13 that the prediction is correct (step S13, “Yes”), the learning/correction unit 24 returns the process to the step S10, and repeats the processes from the step S10. On the other hand, if determined in the step S13 that the prediction is not correct (step S13, “No”), the learning/correction unit 24 proceeds to step S14.

In the step S14, the learning/correction unit 24 acquires correction data for correcting the learned model by the correction unit 241.

More specifically, for example, the learning/correction unit 24 generates an arm control signal for enabling manual operation of the robot arm apparatus 10 in response to the start trigger signal received from the input unit 26, and passes the generated arm control signal to the robot arm apparatus 10. In response to the arm control signal, the operation mode of the robot arm apparatus 10 is shifted from an autonomous operation mode to a manually operable mode.

In the manually operable mode, a surgeon manually manipulates the arm unit 11 to correct the position and/or posture of the endoscope device 12 such that the captured image displayed on the display unit 31 includes a desired imaging range. Upon completion of the correction of the position and/or posture of the endoscope device 12, the surgeon instructs the operation unit 30 to restart the autonomous operation by the robot arm apparatus 10. The input unit 26 outputs an end trigger signal to the learning/correction unit 24 in response to the operation to the operation unit 30.

The learning/correction unit 24 uses the learning unit 240 to pass, when receiving the end trigger signal from the input unit 26, that is, the trigger signal next to the start trigger signal received in the above-described step S13, the input information sₜ at the time of receiving the end trigger signal to the correction unit 241. Thus, the correction unit 241 acquires correction data for correcting the learned model. Further, the correction unit 241 acquires the learned model stored in the storage unit 25.

In the next step S15, the correction unit 241 corrects the learned model acquired from the storage unit 25 based on the correction data acquired in the step S14. The correction unit 241 overwrites the learned model before correction stored in the storage unit 25 by the corrected learned model.

More specifically, the correction unit 241 weights each of the learners 600₁, 600₂, . . . , 600ₙ included in the acquired learned model based on the correction data. In the weighting, the correction unit 241 gives a penalty weight, for example, a larger weight, to the learner (prediction model) that outputs the improper position with respect to the position of the endoscope device 12, and boosts the learner. In other words, learning is performed so that correct answer data can be obtained by considering the data that outputs the improper position as important. As described with reference to FIG. 15, the weighted sum of the learners (prediction model) is the output of the learning model 60, that is, the corrected learned model. Specific examples of weighting will be described below.

After correcting and overwriting the learned model in the step S15, the learning/correction unit 24 returns the process to the step S11, shifts the operation mode of the robot arm apparatus 10 from the manually operable mode to the autonomous operation mode, and executes prediction based on the corrected learned model and drive control of the robot arm apparatus 10.

A specific example of weighting performed in the step S15 by the correction unit 241 will be described. The input information sₜ as correction information to be corrected is as follows.

Position of the endoscope device 12 corrected by surgeons (proper position)

Position of the endoscope device 12 considered abnormal by surgeons (improper position)

In this case, for example, a greater weight can be given to the learner (prediction model) that outputs the improper position. In addition, weighting may be applied to the learner related to the zoom amount of the endoscope device 12 in the proper position or the improper position, or the captured image itself. Further, when other information is used as the input information sₜ weighting may be performed on the learner related to the other information according to the proper position or the improper position.

The correction unit 241 can further perform weighting according to a trigger signal. For example, the correction unit 241 can use the time from the start of the autonomous operation to the output of the start trigger signal as the correction information.

The correction unit 241 can further perform weighting according to a correct answer label indicating a correct answer or an incorrect answer. In the above description, although the correction unit 241 obtains the correct answer label at the time when the autonomous operation is stopped and immediately before the autonomous operation is restarted, the correction unit is not limited to this example. For example, it is conceivable that a correct answer label is acquired according to the result of comparing each of the input information sₜ at the time when the autonomous operation is stopped in response to the start trigger signal with the correction information (each of the input information sₜ₊₁) at the time when the end trigger signal is outputted from the input unit 26.

Further, the correction unit 241 is not limited to the correct answer label represented by a binary value of 0 or 1, and may perform weighting according to the reliability r taking a value of 0≤r≤1, for example. It is conceivable that the reliability r may be obtained for each of the learners 600₁ to 600ₙ, for example, as a value corresponding to the above result of comparing each of the input information sₜ with each of the correction information (input information sₜ₊₁).

The correction unit 241 can further weight each of the learners 600₁ to 600ₙ to the weighted prediction model itself. For example, the assumption is made that the configuration having each of the learners 600₁ to 600ₙ described with reference to FIG. 15 is a prediction model, and that the learning model 60 has a layer structure including a plurality of the prediction models as in each of the learners 600₁ to 600ₙ in FIG. 15. In the structure, weighting is applied to each of the prediction models or each of the learners 600₁ to 600ₙ included in each of the prediction models as weak learners. Further, applying weighting to a weakly supervised feature amount in each weak learner is also conceived.

Thus, weighting the parameters relating to the samples to, for example, each of the learners 600₁ to 600ₙ allows the relearning of the learned model by online learning to be performed efficiently.

In the above description, although the existing learned model is corrected by weighting the prediction model, the relearning is not limited to this example, and a new prediction model including a proper position of the endoscope device 12, for example, may be generated.

The process according to the flowchart in FIG. 16 will be described with more specific examples. In the medical imaging system 1a, the robot arm apparatus 10 is autonomously operated based on a previously generated learned model, and a captured image or a surgical field image based on the captured image captured by the endoscope device 12 supported by the arm unit 11 is displayed on the display unit 31. The surgeon operates the surgical instrument while looking at the image displayed on the display unit 31 to perform the surgery.

When the surgeon notices an unnatural imaging position in the image displayed on the display unit 31, the surgeon instructs the operation unit 30 to stop the autonomous operation and to start manual correction of the position of the endoscope device 12. The input unit 26 outputs a start trigger signal to the learning/correction unit 24 in response to the operation (step S13 in FIG. 16, “No”).

In response to the start trigger signal, the learning/correction unit 24 determines that the current position of the endoscope device 12 is an improper position, and gives an improper label (or an incorrect answer label) to the prediction model that outputs the improper position. Further, the learning/correction unit 24 outputs an arm control signal for stopping the autonomous operation and enabling manual operation. Thus, the operation mode of the robot arm apparatus 10 is shifted from the autonomous operation mode to the manually operable mode.

The surgeon manually corrects the position of the endoscope device 12 to the correct position while checking the captured image displayed on the display unit 31. When the position correction is completed, the surgeon performs an operation for indicating the position correction to the operation unit 30. The input unit 26 outputs an end trigger signal to the learning/correction unit 24 in response to the operation.

In response to the end trigger signal, the learning/correction unit 24 acquires the current position of the endoscope device 12 (step S14 in FIG. 16), determines that the acquired position is a proper position, and gives a proper label (or a correct answer label). For example, the learning/correction unit 24 gives a proper label to the prediction model that outputs a position close to the proper position.

The learning/correction unit 24 corrects the prediction model based on the label given to the prediction model (FIG. 16, step S15). For example, the learning/correction unit 24 gives a penalty weight to the prediction model to which an improper label is given, and increases the weight of the prediction model to which a proper label is given. The learning/correction unit 24 may generate a new prediction model based on the label given to the prediction model. The learning/correction unit 24 determines an output based on the weight given to each prediction model and each prediction model.

### 2-4-3. Overview of Surgery when Medical Imaging System according to Embodiment is Applied

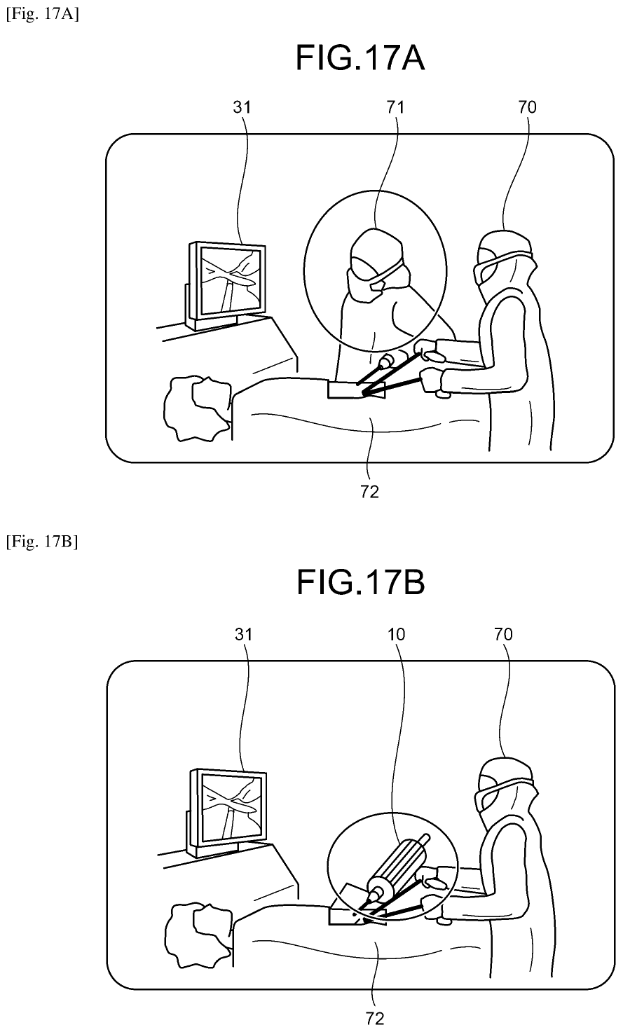

The surgery performed when the medical imaging system 1a according to the embodiment is applied will be then described schematically. FIG. 17A is a diagram schematically illustrating a surgery using an endoscope system according to the existing technique. In the existing technique, at the time of surgery of a patient 72, a surgeon 70 who actually performs the surgery using surgical instruments and an assistant (scopist) 71 who operates the endoscope device must stay beside the patient 72. The surgeon 70 performs the surgery while checking a surgical field image captured by the endoscope device operated by the assistant 71 on the display unit 31.

FIG. 17B is a diagram schematically illustrating a surgery performed when the medical imaging system 1a according to the embodiment is applied. As described above, in the medical imaging system 1a according to the embodiment, the robot arm apparatus 10 including the arm unit 11 on which the endoscope device 12 is supported operates autonomously based on the learned model. The surgeon 70 stops the autonomous operation when an unnatural or abnormal surgical field image displayed on the display unit 31 is recognized, and can manually correct the position of the endoscope device 12. The medical imaging system 1a relearns the learned model based on the corrected position, and restarts the autonomous operation of the robot arm apparatus 10 based on the relearned learned model.

Therefore, the robot arm apparatus 10 can perform an autonomous operation with higher accuracy, and eventually, as illustrated in FIG. 17B, it will be possible to perform a surgery in which the robot arm apparatus 10 is responsible for capturing images with the endoscope device 12, and only the surgeon 70 stays beside the patient 72. Thus, the assistant 71 is not required to stay beside the patient 72, which allows for a wider area around the patient 72.

Further, specific examples of application of the medical imaging system 1a according to the embodiment include the following.

Specific example (1): A surgeon confirms an unnatural autonomous operation of the endoscope device 12 during a surgery 1, and the surgeon stops the autonomous operation, performs slight correction on the spot, and restarts the autonomous operation. In the surgery after the restart of the autonomous operation, the unnatural autonomous operation has not occurred.

Specific example (2): A surgeon confirms the unnatural movement of the endoscope device 12 during the simulation work before the surgery and corrects the movement by voice (speech correction will be discussed below), and then the unnatural movement has not occurred during the actual surgery.

Specific example (3): The surgical pattern of a surgeon A is generally different from that of a surgeon B. Therefore, when the surgeon A uses the learned model learned based on the surgical operation of the surgeon B in performing the surgery, the trajectory of the endoscope device 12 is different from the trajectory desired by the surgeon A. Even in such a case, the trajectory of the endoscope device 12 desired by the surgeon A can be adapted intraoperatively or during preoperative training.

When the surgical targets are different, the surgical pattern may be different and the trajectory of the endoscope device 12 desired by the surgeon may be different. Even in such a case, the surgical pattern learned by the learned model can be used. Alternatively, the surgical targets can be categorized, and learned models for each category can be generated.

### 2-5. Variation of Embodiment

A variation of the embodiment will be then described. In the medical imaging system 1a according to the above-described embodiment, although the input unit 26 has been described as outputting the start trigger signal and the end trigger signal in response to an operation to the operation unit 30, the input unit is not limited to this example. The variation of the embodiment is an example in which the input unit 26 outputs a start trigger signal and an end trigger signal in response to voice.

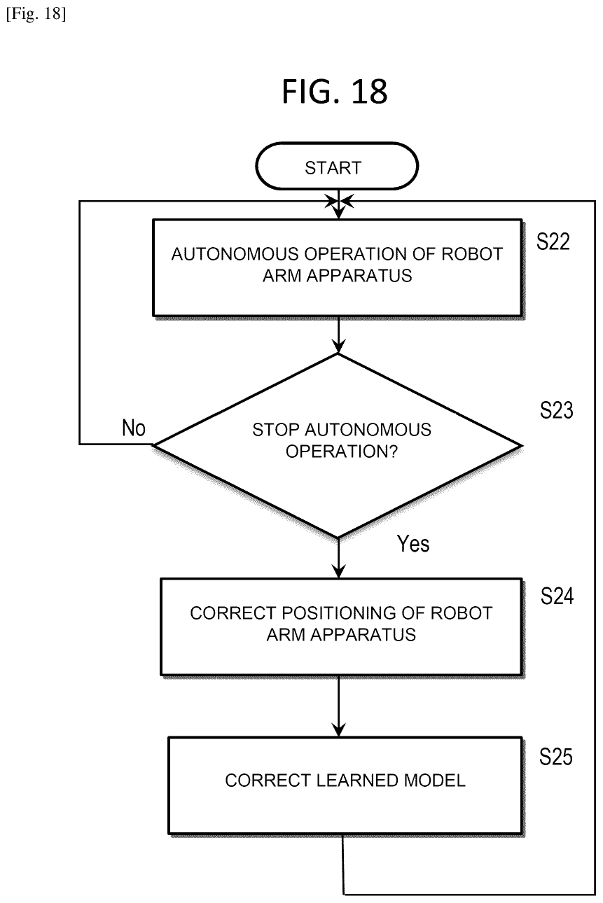

FIG. 18 is a flowchart illustrating an example of operations associated with the surgery performed using the medical imaging system according to the embodiment. The flowchart may be representative of the operations performed for the surgery described with respect to FIG. 17B.

As noted above, the robot arm apparatus, which includes the arm unit 11 on which the endoscope device 12 is supported (which may be referred to herein as a medical articulating arm) can be operating autonomously, for instance, in an autonomous mode, based on a learned model (step S22 in FIG. 18).

A command to stop the autonomous mode may be received, for instance, from a surgeon performing surgery (actual or simulated) using the medical imaging system 1a (FIG. 18, step S23). The autonomous operation may be stopped when an unnatural or abnormal surgical field image displayed on the display unit 31 is recognized, for instance, by the surgeon. Stopping the autonomous mode may place the medical imaging system 1a in a manual mode, for manual operation and/or manipulation of the the arm unit 11 (and the endoscope device 12).

The positioning of the arm unit 11 (and the endoscope device 12) can be corrected, for instance, by the surgeon (FIG. 18, step S24). Such correction may be by way of physically contacting the arm unit 11 to change positioning or by way of a voice command to change positioning of the arm unit 11. The positioning of the arm unit 11 before and after correction may be saved as correction data for providing as input(s) to the current learned model. Correction inputs can be received by the control unit 20a, for instance, by the input unit 26.

The learned model can be corrected using correction data (step S25, FIG. 18). The medical imaging system 1a can relearn, i.e., correct, the learned model based on the correction data. The learning/correction unit 24 can perform the processing to correct the learned model. For instance, the weighting processing, such as described above, can be implemented to correct the learned model based on the correction data.

Once the learned model has been corrected, the autonomous operation can be restarted and the arm unit 11 (and the endoscope device 12) can be controlled according to the corrected learned model. Thus, feedback to the arm unit 11 may be controlled by the learning model of the learning/correction unit 24 of the control unit 20a.

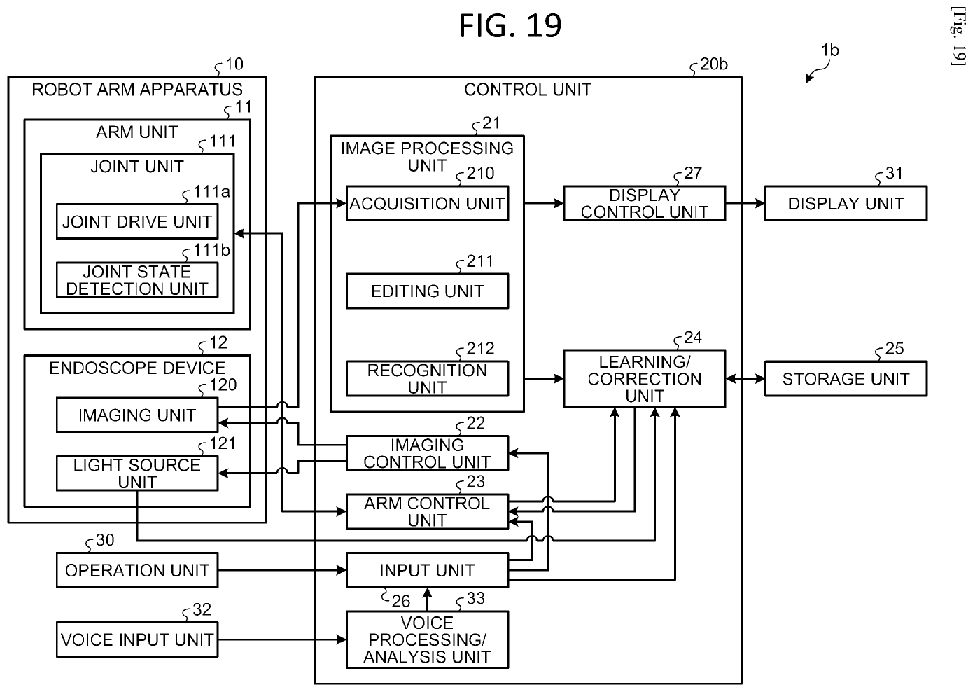

FIG. 19 is a functional block diagram illustrating an example of a functional configuration of a medical imaging system corresponding to a trigger signal outputted by voice applicable to the embodiment. The medical imaging system 1b illustrated in FIG. 19 has a voice input unit 32 added to the medical imaging system 1a described in FIG. 7, and a control unit 20b has a voice processing/analysis unit 33 added to the control unit 20a in the medical imaging system 1a described in FIG. 7.

In the medical imaging system 1b, the voice input unit 32 is, for example, a microphone, and collects voice and outputs an analog voice signal. The voice signal outputted from the voice input unit 32 is inputted to the voice processing/analysis unit 33. The voice processing/analysis unit 33 converts an analog voice signal inputted from the voice input unit 32 into a digital voice signal, and performs voice processing such as noise removal and equalization processing on the converted voice signal. The voice processing/analysis unit 33 performs voice recognition processing on the voice signal subjected to the voice processing to extract a predetermined utterance included in the voice signal. As the voice recognition processing, known techniques such as a hidden Markov model and a statistical technique can be applied.

The voice processing/analysis unit 33, when utterance (for example, “stop” and “suspend”) for stopping the autonomous operation of the arm unit 11 is extracted from the voice signal, inputs the extracted signal to the input unit 26. The input unit 26 outputs a start trigger signal in response to the notification. Further, the voice processing/analysis unit 33, when utterance (for example, “start” and “restart”) for restarting the autonomous operation of the arm unit 11 is extracted from the voice signal, inputs the extracted signal to the input unit 26. The input unit 26 outputs an end trigger signal in response to the notification.

Outputting the trigger signal using the voice allows, for example, a surgeon to instruct to stop or restart the autonomous operation of the arm unit 11 without releasing his/her hand from a surgical instrument.

Further, the medical imaging system 1b can correct the position and/or posture of the endoscope device 12 by voice. For example, when the operation mode of the robot arm apparatus 10 is a manually operable mode and a predetermined keyword (for example, “to the right”, “a little to the left”, and “upwards”) for correcting the position and/or posture of the endoscope device 12 is extracted from the voice signal inputted from the voice input unit 32, the voice processing/analysis unit 33 passes an instruction signal corresponding to each of the keywords to the arm control unit 23. The arm control unit 23 executes drive control of the arm unit 11 in response to the instruction signal passed from the voice processing/analysis unit 33. Thus, the surgeon can correct the position and/or posture of the endoscope device 12 without releasing his/her hand from the surgical instrument.

### 2-6. Effect of Embodiment

The effect of the embodiment will be then described. The effect of the embodiment will be first described in comparison with the existing technique.

The above-mentioned Patent Literature 1 discloses a technique for automatic operation of an endoscope. According to the technique of Patent Literature 1, there is a part related to the present disclosure in terms of feedback of control parameters. However, in the technique of Patent Literature 1, the control unit is the main unit, and only the control input is used as the external input. Therefore, there is a possibility of responding to differences in surgical operators or slight differences in surgery. In addition, since the control unit is the main unit and the feedback to the control unit is the answer, it is difficult to provide correct answer data.

On the other hand, in the present disclosure, the position and/or posture of the endoscope device 12 is manually corrected based on the judgment of the surgeon. Therefore, even a response to slight differences in surgery disclosed in Patent Literature 1 can be corrected on the spot. Further, since the unnaturalness or abnormality of the trajectory of the endoscope device 12 is determined by the surgeon and the position and/or posture of the endoscope device 12 is corrected, it is easy to provide correct answer data.

Further, the Patent Literature 2 discloses a technique for integrating sequential images for robotic surgery. The Patent Literature 2 is an image-based approach to image integration and does not disclose an autonomous operation of a robot holding an endoscope, but discloses a system for recognition and prediction.

On the other hand, the present disclosure relates to the autonomous operation of the robot arm apparatus 10 for supporting the endoscope device 12 and is not dependent on image.

Thus, the technique disclosed in the present disclosure is clearly different from the techniques disclosed in Patent Literatures 1 and 2.

In addition, according to the embodiment and its variation, the position and/or posture of the endoscope device 12 may be provided by a position and/or posture corresponding to the position of the surgical instrument actually being performed by the surgeon in the surgery, rather than a heuristic position and/or posture.

Further, according to the embodiment and its variation, the insufficiency of the control by the learned model at a certain point of time can be corrected in the actual situation where the surgeon uses the surgical instrument. It is also possible to design such that improper output is not repeated.

In addition, according to the embodiment and its variation, the position and/or posture of the endoscope device 12, which is appropriate for each surgeon, can be optimized by the correction unit 241. Thus, it is possible to handle a surgery by multiple surgeons.

In addition, according to the embodiment and its variation, the autonomous operation of the robot arm apparatus 10 is stopped based on the judgment of the surgeon, the position and/or posture of the endoscope device 12 is manually corrected, and the autonomous operation based on the learned model reflecting the correction is restarted after the correction is completed. Therefore, the correction can be performed in real time, and the correction can be performed immediately when the surgeon feels a sense of incongruity in the trajectory of the endoscope device 12.

In addition, according to the embodiment and its variation, since the autonomous operation is hardly affected by the captured image, the lighting to the surgical site and the influence of the imaging unit 120 in the endoscope device 12 can be reduced.

The variation of the embodiment also allows for voice response, allowing a surgeon to have a smooth interaction with the robot arm apparatus 10.

Further, the embodiment and its variation can also estimate the position of the surgical instrument from the captured image, eliminating the process of measuring the position of the surgical instrument.

### 2-7. Application Example of Techniques of Present Disclosure

Although the technique of the present disclosure has been described above as being applicable to medical imaging systems, the technique is not limited to this example. The technique according to the present disclosure may be considered to be synonymous with a technique for correcting a captured image (streaming video) by providing a correct answer label based on an evaluation by a user for a robot performing autonomous operation.

Therefore, the technique according to the present disclosure is applicable to a system for photographing a moving image by autonomous operation, such as a camera work for photographing a movie, a camera robot for watching a sports game, or a drone camera. Applying the technique of the present disclosure to such a system allows, for example, a skilled photographer or operator to sequentially customize the autonomous operation according to his or her own operation feeling.

As an example, in the input/output to/from the camera work for movie shooting, the prediction model (learning model) is as follows.

Input information: camera captured image, global position, velocity, acceleration, and zoom amount at time t

Output information: camera captured image, global position, velocity, acceleration, and zoom amount at time t+1

The corrected model is as follows.

Input information: camera captured image, global position, velocity, acceleration, and zoom amount before and after correction, and correct answer labels before and after correction

Output information: each predictor (learner) and weights given to each predictor, or weighted prediction model

Further, when applying the technique of the present disclosure to a camera robot for watching sports, generating a prediction model for each sport event such as basketball and soccer is further conceived. In such a case, the camera work can be changed by sequentially correcting the prediction model according to the actual accident or the situation of the team at different times.

Some or all of the units described above may be implemented fully or partially using circuitry. For instance, the control unit 20a and/or the control unit 20b may be implemented fully or partially using circuitry. Thus, such control unit(s) may be referred to or characterized as control circuitry. Each of such control unit(s) may also be referred to herein as a controller or a processor. Likewise, processing operations or functions, for instance, of the control unit 20a (or 20b) may be implemented fully or partially using circuitry. For instance, processing performed by the learning/correction unit 24 may be implemented fully or partially using circuitry. Thus, such unit(s) may be referred to or characterized as processing circuitry. Examples of processors according to embodiments of the disclosed subject matter include a micro-controller unit (MCU), a central processing unit (CPU), a digital signal processor (DSP), or the like. The control unit 20a (or 20b) may have or be operatively coupled to non-transitory computer-readable memory, which can be a tangible device that can store instructions for use by an instruction execution device (e.g., a processor or multiple processors, such as distributed processors). The non-transitory storage medium may be, for example, but is not limited to, an electronic storage device, a magnetic storage device, an optical storage device, an electromagnetic storage device, a semiconductor storage device, or any appropriate combination of these devices.

Note that the effects described herein are merely examples and are not limited thereto, and other effects may be provided.

Note that the present technique may have the following configuration.

1a, 1b MEDICAL IMAGING SYSTEM

10 ROBOT ARM APPARATUS

11 ARM UNIT

12 ENDOSCOPE DEVICE

13, 5003 LENS BARREL

20a, 20b CONTROL UNIT

21 IMAGE PROCESSING UNIT

22 IMAGING CONTROL UNIT

23 ARM CONTROL UNIT

24 LEARNING/CORRECTION UNIT

25 STORAGE UNIT

26 INPUT UNIT

30 OPERATION UNIT

31 DISPLAY UNIT

32 VOICE INPUT UNIT

33 VOICE PROCESSING/ANALYSIS UNIT

60 LEARNING MODEL

111 JOINT UNIT

111₁, 111₁₁ FIRST JOINT UNIT

111₂, 111₁₂ SECOND JOINT UNIT

111₃, 111₁₃ THIRD JOINT UNIT

111₄ FOURTH JOINT UNIT

111a JOINT DRIVE UNIT

111b JOINT STATE DETECTION UNIT

120 IMAGING UNIT

121 LIGHT SOURCE UNIT

240 LEARNING UNIT

241 CORRECTION UNIT

600₁, 600₂, 600ₙ LEARNER

601 PREDICTOR

Embodiments of the disclosed subject matter can also be according to the following parentheticals:

### 1

A medical arm system comprising: a medical articulating arm provided with an endoscope at a distal end portion thereof; and control circuitry configured to predict future movement information for the medical articulating arm using a learned model generated based on learned previous movement information from a prior non-autonomous trajectory of the medical articulating arm performed in response to operator input and using current movement information for the medical articulating arm, generate control signaling to autonomously control movement of the medical articulating arm in accordance with the predicted future movement information for the medical articulating arm, and autonomously control the movement of the medical articulating arm in accordance with the predicted future movement information for the medical articulating arm based on the generated control signaling.

### 2

The medical arm system according to (1), wherein the previous movement information and the future movement information for the medical articulating arm includes position and/or posture of the endoscope of the medical articulating arm.

### 3

The medical arm system according to (1) or (2), wherein the control circuitry is configured to determine whether the predicted current movement information for the medical articulating arm is correct, and correct a previous learned model to generate said learned model.

### 4

The medical arm system according to any one of (1) to (3), wherein the control circuitry is configured to correct the previous learned model based on the determination indicating that the predicted current movement information for the medical articulating arm is incorrect.

### 5

The medical arm system according to any one of (1) to (4), wherein the determination of whether the predicted current movement information for the medical articulating arm is correct is based on the operator input, the operator input being a manual manipulation of the medical articulating arm by an operator of the medical arm system to correct position and/or posture of the medical articulating arm.

### 6

The medical arm system according to any one of (1) to (5), wherein the control circuitry is configured to generate the learned model based on the learned previous movement information from the prior non-autonomous trajectory of the medical articulating arm performed in response to the operator input at an operator input interface.

### 7

The medical arm system according to any one of (1) to (6), wherein input information to the learned model includes the current movement information for the medical articulating arm, the current movement information for the medical articulating arm including position and/or posture of the endoscope of the medical articulating arm and position and/or posture of another surgical instrument associated with a procedure to be performed using the medical arm system.

### 8

The medical arm system according to any one of (1) to (7), wherein the control circuitry predicts the future movement information for the medical articulating arm using the learned model according to equations (i) and (ii):

sₜ₊₁=f(sₜ) (i)

yₜ=g(sₜ) (ii),

where s is input to the learned model, y is output from the learned model, t is time, f(sₜ) is a function of the input sₜ₊₁ at time t+1, and g(sₜ) is a function of the output of the learned model at time t.

### 9

The medical arm system according to any one of (1) to (8), wherein the control circuitry is configured to switch from an autonomous operation mode to a manual operation mode in association with a trigger signal to correct the learned model.

### 10

The medical arm system according to any one of (1) to (9), wherein the learned model implemented by the control circuitry includes a plurality of different learners having respective outputs provided to a same predictor, and wherein the control circuitry is configured to correct the learned model by weighting each of the plurality of different learners based on acquired correction data associated with the autonomous control of the movement of the medical articulating arm and manual control of the medical articulating arm.

### 11

The medical arm system according to any one of (1) to (10), wherein for the weighting the control circuitry gives greater importance to one or more of the different learners that outputs improper position with respect to position of the endoscope on the medical articulating arm.

### 12

The medical arm system according to any one of (1) to (11), wherein the control circuitry applies the weighting in relation to either a zoom amount of the endoscope in proper/improper position or an image captured by the endoscope.

### 13

The medical arm system according to any one of (1) to (12), wherein the correction data for the weighting includes timing from a start of an autonomous operation to output of a start trigger signal associated with switching from the autonomous control to the manual control.

### 14

The medical arm system according to any one of (1) to (13), wherein the weighting is performed according to correct answer labeling and/or reliability of the correct answer labeling for each of the different learners.

### 15

The medical arm system according to any one of (1) to (14), wherein the weighting includes weighting of a weighted prediction model.

### 16

The medical arm system according to any one of (1) to (15), wherein control circuitry is configured to determine whether the predicted current movement information for the medical articulating arm is correct, the determination of whether the predicted current movement information for the medical articulating arm is correct is based on the operator input, the operator input being a voice command of an operator of the medical arm system to correct position and/or posture of the medical articulating arm.

### 17

The medical arm system according to any one of (1) to (16), wherein the learned model is specific to a particular operator providing the operator input at an operator input interface.

### 18

A method regarding an endoscope system comprising: providing, using a processor of the endoscope system, previous movement information regarding a prior trajectory of a medical articulating arm of the endoscope system performed in response to operator input; and generating, using the processor of the endoscope system, a learned model to autonomously control the medical articulating arm based on an input in the form of the previous movement information regarding the prior trajectory of the medical articulating arm provided using the processor and an input in the form of current movement information for the medical articulating arm.

### 19

The method according to (18), wherein said generating includes updating a previous learned model to generate the learned model using acquired correction data associated with previous autonomous control of movement of the medical articulating arm compared to subsequent manual control of the medical articulating arm.

### 20

The method according to (18) or (19), wherein said generating includes: determining whether predicted current movement information for the medical articulating arm predicted using a previous learned model was correct; and correcting the previous learned model to generate said learned model.

### 21

The method according to any one of (18) to (20), wherein said correcting the previous learned model is based on said determining indicating that the predicted current movement information for the medical articulating arm was incorrect.

### 22

The method according to any one of (18) to (21), wherein said determining whether the predicted current movement information was correct is based on the operator input, the operator input being a manual manipulation of the medical articulating arm by an operator to correct position and/or posture of an endoscope of the endoscope system.

### 23

The method according to any one of (18) to (22), further comprising switching from an autonomous operation mode to a manual operation mode in association with a trigger signal to correct the learned model.

### 24

The method according to any one of (18) to (23), wherein said generating includes weighting a plurality of different learners of a previous learned model to generate the learned model.

### 25

The method according to any one of (18) to (24), wherein said weighting the plurality of different learners is based on acquired correction data associated with autonomous control of the movement of the medical articulating arm and subsequent manual control of the medical articulating arm.

### 26

The method according to any one of (18) to (25), wherein the correction data for said weighting includes timing from a start of an autonomous operation to output of a start trigger signal associated with switching from autonomous control to manual control of the endoscope system.

### 27

The method according to any one of (18) to (26), wherein said weighting gives greater weight to one or more of the different learners that outputs improper position with respect to position of an endoscope of the endoscope system.

### 28

The method according to any one of (18) to (27), wherein said weighting is applied in relation to either a zoom amount of an endoscope of the endoscope system in proper/improper position or an image captured by the endoscope.

### 29

The method according to any one of (18) to (28), wherein said weighting is performed according to correct answer labeling and/or reliability of the correct answer labeling for each of the different learners.

### 30

The method according to any one of (18) to (29), wherein said weighting includes weighting of a weighted prediction model.

### 31

The method according to any one of (18) to (30), wherein said generating includes determining whether predicted current movement information for the medical articulating arm predicted is correct based on the operator input, the operator input being a voice command of an operator of the endoscope system to correct position and/or posture of an endoscope of the endoscope system, and wherein said generating is performed as part of a simulation performed prior to a surgical procedure using the endoscope system.

### 32

The method according to any one of (18) to (31), wherein said generating includes acquiring correction data associated with autonomous control of the movement of the medical articulating arm and subsequent manual control of the medical articulating arm.

### 33

The method according to any one of (18) to (32), wherein an output of the generated learned model includes a predicted position and/or posture of the medical articulating arm.

### 34

The method according to any one of (18) to (33), wherein the previous movement information regarding the prior trajectory of a medical articulating arm is provided from memory of the endoscope system to the controller.

### 35

The method according to any one of (18) to (34), wherein the previous movement information includes position and/or posture of the medical articulating arm.

### 36

A method of controlling a medical articulating arm provided with an endoscope at a distal end portion thereof, the method comprising: predicting, using a controller, future movement information for the medical articulating arm using a learned model generated based on learned previous movement information from a prior non-autonomous trajectory of the medical articulating arm performed in response to operator input and using current movement information for the medical articulating arm; generating, using the controller, control signaling to autonomously control movement of the medical articulating arm in accordance with the predicted future movement information for the medical articulating arm; and autonomously controlling, using the controller, the movement of the medical articulating arm in accordance with the predicted future movement information for the medical articulating arm based on the generated control signaling.

### 37

The method according to (36), wherein the previous movement information and the future movement information for the medical articulating arm includes position and/or posture of the endoscope of the medical articulating arm.

### 38

The method according to (36) or (37), further comprising: determining, using the controller, whether the predicted current movement information for the medical articulating arm is correct; and correcting, using the controller, a previous learned model to generate said learned model.

### 39

The method according to any one of (36) to (38), wherein said correcting is based on said determining indicating that the predicted current movement information for the medical articulating arm is incorrect.

### 40

The method according to any one of (36) to (39), wherein the determination of whether the predicted current movement information for the medical articulating arm is correct is based on the operator input, the operator input being a manual manipulation of the medical articulating arm by an operator of the medical arm system to correct position and/or posture of the medical articulating arm.

### 41

The method according to any one of (36) to (40), wherein said generating the learned model is based on the learned previous movement information from the prior non-autonomous trajectory of the medical articulating arm performed in response to the operator input at an operator input interface.

### 42

The method according to any one of (36) to (41), wherein input information to the learned model includes the current movement information for the medical articulating arm, the current movement information for the medical articulating arm including position and/or posture of the endoscope of the medical articulating arm and position and/or posture of another surgical instrument associated with a procedure to be performed using the medical arm system.

### 43

The method according to any one of (36) to (42), wherein said predicting the future movement information for the medical articulating arm uses the learned model according to equations (1) and (2):

sₜ₊₁=f(sₜ) (1)

yₜ=g(sₜ) (2),

where s is input to the learned model, y is output from the learned model, t is time, f(sₜ) is a function of the input sₜ₊₁ at time t+1, and g(sₜ) is a function of the output of the learned model at time t.

### 44

The method according to any one of (36) to (43), further comprising switching, using the controller, from an autonomous operation mode to a manual operation mode in association with a trigger signal to correct the learned model.

### 45

The method according to any one of (36) to (44), wherein the learned model includes a plurality of different learners having respective outputs provided to a same predictor, and wherein said correcting the learned model includes weighting each of the plurality of different learners based on acquired correction data associated with the autonomous control of the movement of the medical articulating arm and manual control of the medical articulating arm.

### 46

The method according to any one of (36) to (45), wherein for said weighting gives greater importance to one or more of the different learners that outputs improper position with respect to position of the endoscope on the medical articulating arm.

### 47

The method according to any one of (36) to (46), wherein said weighting is applied in relation to either a zoom amount of the endoscope in proper/improper position or an image captured by the endoscope.

### 48

The method according to any one of (36) to (47), wherein the correction data for the weighting includes timing from a start of an autonomous operation to output of a start trigger signal associated with switching from the autonomous control to the manual control.

### 49

The method according to any one of (36) to (48), wherein said weighting is performed according to correct answer labeling and/or reliability of the correct answer labeling for each of the different learners.

### 50

The method according to any one of (36) to (49), wherein said weighting includes weighting of a weighted prediction model.

### 51

The method according to any one of (36) to (50), further comprising determining whether the predicted current movement information for the medical articulating arm is correct based on the operator input, the operator input being a voice command of an operator to correct position and/or posture of the medical articulating arm.

### 52

The method according to any one of (36) to (51), wherein the learned model is specific to a particular operator providing the operator input at an operator input interface.

### 53

A system comprising: a medical articulating arm; an endoscope operatively coupled to the medical articulating arm; and processing circuitry configured to provide previous movement information regarding a prior trajectory of a medical articulating arm of the endoscope system performed in response to operator input, and generate a learned model to autonomously control the medical articulating arm based on an input in the form of the previous movement information regarding the prior trajectory of the medical articulating arm provided using the processor and an input in the form of current movement information for the medical articulating arm.

### 54

The system according to (53), wherein the processing circuitry is configured to update a previous learned model to generate the learned model using acquired correction data associated with previous autonomous control of movement of the medical articulating arm compared to subsequent manual control of the medical articulating arm.

### 55

The system according to (53) or (54), wherein the processing circuitry, to generate the learned model, is configured to: determine whether predicted current movement information for the medical articulating arm predicted using a previous learned model was correct; and correct the previous learned model to generate said learned model.

### 56

The system according to any one of (53) to (55), wherein the processing circuitry corrects the previous learned model based on the determination indicating that the predicted current movement information for the medical articulating arm was incorrect.

### 57

The system according to any one of (53) to (56), wherein the processing circuitry determines whether the predicted current movement information was correct based on the operator input, the operator input being a manual manipulation of the medical articulating arm by an operator to correct position and/or posture of an endoscope of the endoscope system.

### 58

The system according to any one of (53) to (57), wherein the processing circuitry is configured to switch from an autonomous operation mode to a manual operation mode in association with a trigger signal to correct the learned model.

### 59

The system according to any one of (53) to (58), wherein the processing circuitry generates the learned model by weighting a plurality of different learners of a previous learned model to generate the learned model.

### 60

The system according to any one of (53) to (59), wherein the processing circuitry weights the plurality of different learners based on acquired correction data associated with autonomous control of the movement of the medical articulating arm and subsequent manual control of the medical articulating arm.

### 61

The system according to any one of (53) to (60), wherein the correction data for the weighting includes timing from a start of an autonomous operation to output of a start trigger signal associated with switching from autonomous control to manual control of the endoscope system.

### 62

The system according to any one of (53) to (61), wherein the processing circuitry, for the weighting, gives greater weight to one or more of the different learners that outputs improper position with respect to position of an endoscope of the endoscope system.

### 63

The system according to any one of (53) to (62), wherein the processing circuitry applies the weighting in relation to either a zoom amount of an endoscope of the endoscope system in proper/improper position or an image captured by the endoscope.

### 64

The system according to any one of (53) to (63), wherein the processing circuitry performs the weighting according to correct answer labeling and/or reliability of the correct answer labeling for each of the different learners.

### 65

The system according to any one of (53) to (64), wherein the weighting includes weighting of a weighted prediction model.

### 66

The system according to any one of (53) to (65), wherein the processing circuitry is configured to, for the generating determine whether predicted current movement information for the medical articulating arm predicted is correct based on the operator input, the operator input being a voice command of an operator of the endoscope system to correct position and/or posture of an endoscope of the endoscope system, and wherein the processing circuitry performs the generation of the learned model as part of a simulation performed prior to a surgical procedure using the endoscope system.

### 67

The system according to any one of (53) to (66), wherein the processing circuitry is configured to, for the generating the learned model, acquire correction data associated with autonomous control of the movement of the medical articulating arm and subsequent manual control of the medical articulating arm.

### 68

The system according to any one of (53) to (67), wherein an output of the generated learned model includes a predicted position and/or posture of the medical articulating arm.

### 69

The system according to any one of (53) to (68), wherein the previous movement information regarding the prior trajectory of a medical articulating arm is provided from memory of the endoscope system to the controller.

### 70

The system according to any one of (53) to (69), wherein the previous movement information includes position and/or posture of the medical articulating arm.

### 71

The medical arm system according to any one of (1) to (17), wherein the learned model is an updated learned model updated from first learned previous movement information from a first prior non-autonomous trajectory of the medical articulating arm performed in response to a first operator input to said learned previous movement information from said prior non-autonomous trajectory of the medical articulating arm performed in response to said operator input.

1. A medical arm system comprising:
a medical articulating arm provided with an endoscope at a distal end portion thereof; and
control circuitry configured to
predict future movement information for the medical articulating arm using a learned model generated based on learned previous movement information from a prior non-autonomous trajectory of the medical articulating arm performed in response to operator input and using current movement information for the medical articulating arm,
generate control signaling to autonomously control movement of the medical articulating arm in accordance with the predicted future movement information for the medical articulating arm, and
autonomously control the movement of the medical articulating arm in accordance with the predicted future movement information for the medical articulating arm based on the generated control signaling.

2. The medical arm system according to claim 1, wherein the previous movement information and the future movement information for the medical articulating arm includes position and/or posture of the endoscope of the medical articulating arm.

3. The medical arm system according to claim 1, wherein the control circuitry is configured to
determine whether the predicted current movement information for the medical articulating arm is correct, and
correct a previous learned model to generate said learned model.

4. The medical arm system according to claim 3, wherein the control circuitry is configured to correct the previous learned model based on the determination indicating that the predicted current movement information for the medical articulating arm is incorrect.

5. The medical arm system according to claim 3, wherein the determination of whether the predicted current movement information for the medical articulating arm is correct is based on the operator input, the operator input being a manual manipulation of the medical articulating arm by an operator of the medical arm system to correct position and/or posture of the medical articulating arm.

6. The medical arm system according to claim 1, wherein the control circuitry is configured to generate the learned model based on the learned previous movement information from the prior non-autonomous trajectory of the medical articulating arm performed in response to the operator input at an operator input interface.

7. The medical arm system according to claim 1, wherein input information to the learned model includes the current movement information for the medical articulating arm, the current movement information for the medical articulating arm including position and/or posture of the endoscope of the medical articulating arm and position and/or posture of another surgical instrument associated with a procedure to be performed using the medical arm system.

8. The medical arm system according to claim 1, wherein the control circuitry predicts the future movement information for the medical articulating arm using the learned model according to equations (1) and (2):
sₜ₊₁=f(sₜ) (1)
yₜ=g(sₜ) (2),
where s is input to the learned model, y is output from the learned model, t is time, f(sₜ) is a function of the input sₜ₊₁ at time t+1, and g(sₜ) is a function of the output of the learned model at time t.

9. The medical arm system according to claim 1, wherein the control circuitry is configured to switch from an autonomous operation mode to a manual operation mode in association with a trigger signal to correct the learned model.

10. The medical arm system according to claim 1,
wherein the learned model implemented by the control circuitry includes a plurality of different learners having respective outputs provided to a same predictor, and
wherein the control circuitry is configured to correct the learned model by weighting each of the plurality of different learners based on acquired correction data associated with the autonomous control of the movement of the medical articulating arm and manual control of the medical articulating arm.

11. The medical arm system according to claim 10, wherein for the weighting the control circuitry gives greater importance to one or more of the different learners that outputs improper position with respect to position of the endoscope on the medical articulating arm.

12. The medical arm system according to claim 10, wherein the control circuitry applies the weighting in relation to either a zoom amount of the endoscope in proper/improper position or an image captured by the endoscope.

13. The medical arm system according to claim 10, wherein the correction data for the weighting includes timing from a start of an autonomous operation to output of a start trigger signal associated with switching from the autonomous control to the manual control.

14. The medical arm system according to claim 10, wherein the weighting is performed according to correct answer labeling and/or reliability of the correct answer labeling for each of the different learners.

15. The medical arm system according to claim 10, wherein the weighting includes weighting of a weighted prediction model.

16. The medical arm system according to claim 1, wherein control circuitry is configured to determine whether the predicted current movement information for the medical articulating arm is correct, the determination of whether the predicted current movement information for the medical articulating arm is correct is based on the operator input, the operator input being a voice command of an operator of the medical arm system to correct position and/or posture of the medical articulating arm.

17. The medical arm system according to claim 1, wherein the learned model is specific to a particular operator providing the operator input at an operator input interface.

18. The medical arm system according to claim 1, wherein the learned model is an updated learned model updated from first learned previous movement information from a first prior non-autonomous trajectory of the medical articulating arm performed in response to a first operator input to said learned previous movement information from said prior non-autonomous trajectory of the medical articulating arm performed in response to said operator input.

19. A method regarding an endoscope system comprising:
providing, using a processor of the endoscope system, previous movement information regarding a prior trajectory of a medical articulating arm of the endoscope system performed in response to operator input; and
generating, using the processor of the endoscope system, a learned model to autonomously control the medical articulating arm based on an input in the form of the previous movement information regarding the prior trajectory of the medical articulating arm provided using the processor and an input in the form of current movement information for the medical articulating arm.

20. The method according to claim 19, wherein said generating includes updating a previous learned model to generate the learned model using acquired correction data associated with previous autonomous control of movement of the medical articulating arm compared to subsequent manual control of the medical articulating arm.

21. The method according to claim 19, wherein said generating includes:
determining whether predicted current movement information for the medical articulating arm predicted using a previous learned model was correct; and
correcting the previous learned model to generate said learned model.

22. The method according to claim 21, wherein said correcting the previous learned model is based on said determining indicating that the predicted current movement information for the medical articulating arm was incorrect.

23. The method according to claim 21, wherein said determining whether the predicted current movement information was correct is based on the operator input, the operator input being a manual manipulation of the medical articulating arm by an operator to correct position and/or posture of an endoscope of the endoscope system.

24. The method according to claim 19, further comprising switching from an autonomous operation mode to a manual operation mode in association with a trigger signal to correct the learned model.

25. The method according to claim 19, wherein said generating includes weighting a plurality of different learners of a previous learned model to generate the learned model.

26. The method according to claim 25, wherein said weighting the plurality of different learners is based on acquired correction data associated with autonomous control of the movement of the medical articulating arm and subsequent manual control of the medical articulating arm.

27. The method according to claim 26, wherein the correction data for said weighting includes timing from a start of an autonomous operation to output of a start trigger signal associated with switching from autonomous control to manual control of the endoscope system.

28. The method according to claim 25, wherein said weighting gives greater weight to one or more of the different learners that outputs improper position with respect to position of an endoscope of the endoscope system.

29. The method according to claim 25, wherein said weighting is applied in relation to either a zoom amount of an endoscope of the endoscope system in proper/improper position or an image captured by the endoscope.

30. The method according to claim 25, wherein said weighting is performed according to correct answer labeling and/or reliability of the correct answer labeling for each of the different learners.

31. The method according to claim 25, wherein said weighting includes weighting of a weighted prediction model.

32. The method according to claim 19,
wherein said generating includes determining whether predicted current movement information for the medical articulating arm predicted is correct based on the operator input, the operator input being a voice command of an operator of the endoscope system to correct position and/or posture of an endoscope of the endoscope system, and
wherein said generating is performed as part of a simulation performed prior to a surgical procedure using the endoscope system.

33. The method according to claim 19, wherein said generating includes acquiring correction data associated with autonomous control of the movement of the medical articulating arm and subsequent manual control of the medical articulating arm.

34. The method according to claim 19, wherein an output of the generated learned model includes a predicted position and/or posture of the medical articulating arm.

35. The method according to claim 19, wherein the previous movement information regarding the prior trajectory of a medical articulating arm is provided from memory of the endoscope system to the controller.

36. The method according to claim 19, wherein the previous movement information includes position and/or posture of the medical articulating arm.
